(19) United States
(12) Patent Application Publication
(10) Pub. No.: US 2026/0137400 A1
(43) Pub. Date: May 21, 2026

(54) PLACEMENT VERIFICATION SYSTEMS AND ASSOCIATED METHODS FOR SURGICAL PROCEDURES

(71) Applicant: ARTHREX, INC., Naples, FL (US)

(72) Inventors: Maris Prieditis, Naples, FL (US); Scott William Doody, Bonita Springs, FL (US); Nick Metcalfe, Bonita Springs, FL (US); Steven Jim De Leon, Naples, FL (US); Ryan Charles Keefer, Naples, FL (US)

(21) Appl. No.: 19/386,394

(22) Filed: Nov. 12, 2025

Related U.S. Application Data
(60) Provisional application No. 63/722,140, filed on Nov. 19, 2024.

(51) Int. Cl. A61B 17/17; A61B 17/56

(52) U.S. Cl. CPC A61B 17/1739; A61B 2017/568

(57) ABSTRACT

This disclosure relates to surgical systems, devices and methods for planning and implementing surgical procedures. The systems and methods disclosed herein may be utilized to determine a placement of a surgical instrument relative to an anatomy of a patient, which may be specified in a surgical plan established for the patient.

### CROSS-REFERENCE TO RELATED APPLICATIONS

This application claims the benefit of U.S. Provisional Application No. 63/722,140, which was filed on Nov. 19, 2024 and is incorporated herein by reference in its entirety.

### BACKGROUND

This disclosure relates to surgical systems and methods for planning and implementing surgical procedures, including devices useful for determining instrument placement relative to patient anatomy.

Many bones of the human musculoskeletal system include articular surfaces. The articular surfaces articulate relative to other bones to facilitate different types and degrees of joint movement. The articular surfaces can erode or experience bone loss over time due to repeated use or wear or can fracture as a result of a traumatic impact. These types of bone defects can cause joint instability and pain. An implant may be utilized to restore functionality to the joint. The implant may include an articular surface that may cooperate with an articular surface of an adjacent bone or implant. One or more surgical instruments may be positioned relative to the bone and may be utilized to prepare the bone for implantation.

### SUMMARY

This disclosure relates to systems, devices and methods of performing a surgical procedure. One or more position verification members may be utilized for determining the positioning of a surgical instrument relative to the anatomy of a patient.

An assembly for a surgical procedure according to an implementation may include a guide including a guide body dimensioned to guide a surgical instrument. The guide body may include one or more patient-specific contact surfaces and one or more passageways. The assembly may include one or more position verification members receivable in a respective one of the passageways. The position verification member may include at least one indicator and a verification surface adapted to contact tissue. The at least one indicator may be moveable in a plurality of positions relative to the guide body in response to translation of the respective position verification member along the respective passageway. The positions may include a starting position, a stopping position and an intermediate position between the starting and stopping positions. The intermediate position may be associated with a predetermined placement of the one or more patient-specific contact surfaces relative to an anatomical surface of a patient.

An assembly for a surgical procedure according to an implementation may include a guide including a guide body dimensioned to position a surgical instrument. The guide body may be established by a locating module and a depth stop module. The depth stop module may include one or more standoffs which may be aligned with one or more passageways through the guide body. Each standoff may include a respective patient-specific height. The assembly may include one or more position verification members including a respective indicator and a verification surface adapted to contact tissue. The position verification member may be insertable through the respective passageway to indicate whether the locating module may be positioned on an anatomical surface of a patient in a patient-specific position according to a surgical plan based on a position of the indicator relative to the respective standoff.

A surgical kit according to an implementation may include a surgical instrument including one or more passageways. One or more verification members may include a respective elongated pin and a respective carrier. The elongated pin may have a contact surface and an indicator. The carrier may have a carrier passage dimensioned to receive the respective elongated pin. The elongated pin may be moveable relative to the carrier. The carrier may be adapted to engage the surgical instrument to position the elongated pin in the respective passageway to determine a placement of the surgical instrument on an anatomical surface of a patient based on a position of the indicator.

A surgical kit according to an implementation may include a surgical instrument and one or more position verification members having a respective patient-specific dimension according to a surgical plan. The one or more position verification members may be insertable through respective passageways of the surgical instrument to determine a placement of the surgical instrument on an anatomical surface of a patient.

A method of performing an orthopaedic procedure may include placing a surgical instrument on an anatomical surface of a patient. The method may include positioning at least one position verification member through a respective passageway of the surgical instrument such that the at least one position verification member may extend distally from the passageway to contact the anatomical surface. The at least one position verification member may include an indicator moveable in a plurality of positions. The positions may include a starting position, a stopping position and an intermediate position between the starting and stopping positions. The method may include determining that the surgical instrument is placed in a patient-specific position specified by a surgical plan for the patient in response to the indicator being aligned with an alignment feature in the intermediate position, but determining that a placement of the surgical instrument deviates from the patient-specific position in response to the indicator being misaligned with the alignment feature of the surgical instrument.

A method of performing an orthopaedic procedure according to an implementation may include placing a surgical instrument on an anatomical surface of a patient. The method may include positioning at least one patient-specific position verification members relative to the surgical instrument to contact the anatomical surface. The method may include determining whether the surgical instrument may be placed in a patient-specific position specified by a surgical plan for the patient based on a position of the at least one position verification member relative to the surgical instrument.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 discloses a surgical planning system.

FIG. 2 discloses another implementation of a surgical planning system including a user interface.

FIG. 3 discloses a transfer model relative to an anatomical model in a display window of the user interface of FIG. 2.

FIGS. 4-5 disclose perspective views of a surgical assembly including a transfer guide and a set of position verification members positioned relative to an anatomy of a patient according to an implementation.

FIG. 6 disclosed a perspective view of one of the position verification members of FIGS. 4-5 according to an implementation.

FIG. 7 discloses a partially unassembled view of the position verification member of FIG. 6.

FIGS. 8-9 disclose different placements of the position verification member relative to the anatomy.

FIG. 10 discloses a perspective view of a transfer guide placed on a bone according to another implementation.

FIG. 11 discloses a phantom view of the transfer guide of FIG. 10 and a position verification member in contact with the bone.

FIG. 12 discloses a perspective view of a surgical assembly including a transfer guide and a position verification member according to another implementation.

FIG. 13 discloses a sectional view of the surgical assembly taken along line 13-13 of FIG. 12 including the position verification member in contact with the bone.

FIG. 14 discloses a side view of a surgical assembly including a transfer guide and a position verification member according to another implementation.

FIG. 15 discloses an isolated view of the position verification member of FIG. 14.

FIGS. 16-17 disclose perspective views of a surgical assembly including a transfer guide and a set of position verification member according to another implementation.

FIG. 18 discloses a set of position verification members having different dimensions.

FIGS. 19-20 disclose different positions of the position verification members relative to the transfer guide.

FIG. 21 discloses a verification device including a set of position verification members position relative to a surgical instrument according to an implementation.

FIG. 22 discloses a perspective view of a surgical assembly including a transfer guide and a set of position verification members according to another implementation.

FIG. 23 discloses an exploded view of the transfer guide of FIG. 22.

FIG. 24 discloses a sectional view of the assembly taken along line 24-24 of FIG. 22.

FIG. 25 discloses a perspective view of a surgical assembly including a transfer guide and a set of position verification members according to another implementation.

FIG. 26 discloses a front view of the assembly of FIG. 25.

FIG. 27 discloses an exploded view of the transfer guide of FIG. 25.

FIG. 28 discloses a sectional view of the assembly taken along line 28-28 of FIG. 26.

FIG. 29 discloses a view of selected portions of the assembly of FIG. 25.

FIG. 30 discloses a method of performing a surgical procedure according to an implementation.

Like reference numbers and designations in the various drawings indicate like elements.

### DETAILED DESCRIPTION

This disclosure relates to systems, devices and methods of performing a surgical procedure. Implementations of position verification members and surgical instruments are disclosed. The position verification members may be utilized for determining the positioning of an associated surgical instrument relative to the anatomy of a patient.

Orthopaedic and other surgical procedures may utilize one or more surgical instruments (e.g., transfer devices or guides) to position various surgical devices and/or implants relative to the patient anatomy. The transfer guides may be patient-specific, configurable (e.g., calibrated) and/or standard (e.g., fixed and non-patient specific). The transfer guides may include cut (e.g., drill or resection) guides and other surgical instruments.

The transfer guide may be placed (e.g., seated) on the anatomy. The transfer guide may include one or more (e.g., patient-specific) contact surfaces that may contact the anatomy along respective contact points. In scenarios, the guide and/or tissue at the surgical site may at least partially obstruct visibility of the contact surfaces and/or adjacent surfaces of the anatomy. The transfer guide and/or position verification member(s) may be dimensioned based a placement (e.g., position and/or orientation) of the guide relative to the anatomy, which may be predetermined according to a surgical plan for the respective patient. The position verification member(s) may establish contact with the anatomy at respective contact points, which may be specified in the surgical plan. The position verification member(s) may be operable to indicate a (e.g., correct) placement of the guide, and/or respective portions thereof, on the anatomy associated with the respective contact points.

The surgeon may use the position verification members to determine whether the guide may be properly seated on the anatomy, which may improve accuracy in performing the procedure according to the surgical plan for the patient. The verification members may include one or more indicators. The indicators may be observable at a different line of sight than the contact surfaces, which may assist the surgeon in determining the placement (e.g., seating) of the guide. The disclosed techniques may be utilized to reduce an incision size for placement the guide on the anatomy.

An assembly for a surgical procedure according to an implementation may include a guide including a guide body dimensioned to guide a surgical instrument. The guide body may include one or more patient-specific contact surfaces and one or more passageways. The assembly may include one or more position verification members receivable in a respective one of the passageways. The position verification member may include at least one indicator and a verification surface adapted to contact tissue. The at least one indicator may be moveable in a plurality of positions relative to the guide body in response to translation of the respective position verification member along the respective passageway. The positions may include a starting position, a stopping position and an intermediate position between the starting and stopping positions. The intermediate position may be associated with a predetermined placement of the one or more patient-specific contact surfaces relative to an anatomical surface of a patient.

In any implementations, the one or more passageways may include a first passageway having an opening along the respective patient-specific contact surface.

In any implementations, a spring member may be dimensioned to bias the respective position verification member toward the starting position.

In any implementations, the starting position may be associated with an uncompressed state of the spring member. The stopping position may be associated with a compressed state of the spring member.

In any implementations, the spring member may be captured between the guide body and a flange of the respective position verification member.

In any implementations, the position verification member may include an elongated pin extending between a proximal end and a distal end.

In any implementations, the contact surface may be established adjacent to the distal end. The at least one indicator may include a first indicator established adjacent to the proximal end.

In any implementations, the elongated pin may include a patient-specific length.

In any implementations, the elongated pin may be patient-generic.

In any implementations, the at least one indicator may include a first indicator established along a periphery of the elongated pin.

In any implementations, the at least one indicator may include a second indicator established along the proximal end of the elongated pin. The proximal end of the elongated pin may be situated in the respective passageway in the starting position. The proximal end of the elongated pin may protrude from the passageway in the stopping position. The proximal end of the elongated pin may be substantially flush with a rim of the respective passageway in the intermediate position.

In any implementations, the position verification member may include a carrier having a carrier passage dimensioned to receive the elongated pin. The elongated pin may be moveable relative to the carrier. The carrier may be adapted to engage the guide body when the elongated pin may be inserted through the respective passageway.

In any implementations, the carrier may include at least one viewing aperture along a periphery of the carrier passage. The at least one indicator may include a first indicator that may be at least partially alignable with the at least one viewing aperture to indicate the position of the verification surface.

In any implementations, the first indicator may be alignable with the at least one viewing aperture in the intermediate position but not in at least one of the starting position and the stopping position.

In any implementations, the first indicator may be visually distinct from adjacent portions of the carrier bounding the at least one viewing aperture.

In any implementations, the guide body may include a receptacle extending from a proximal end of the passageway. The receptacle may include a patient-specific depth. The carrier may be at least partially receivable in the receptacle to set the position of the elongated pin relative to the guide body.

In any implementations, the position verification member may include a protrusion extending from a periphery of the elongated pin. The guide body may include a J-shaped channel which may include first, second and third segments. The first and third segments may extend lengthwise along the respective passageway. The second segment may extends circumferentially about the respective passageway to interconnect the first and third segments. The elongated pin may be releasably securable in the respective passageway in response to moving the protrusion in a first direction along the first segment, then rotating the elongated pin in a second direction to cause the protrusion to move along the second segment, and then moving the protrusion in a third direction along the third segment.

In any implementations, a wall along the second segment may be dimensioned to bound the stopping position. A wall along a terminal end of the third segment may be dimensioned to bound the starting position.

In any implementations, the assembly may include one or more patient-specific standoffs adjacent the respective one or more passageways. The standoff may be dimensioned such that the proximal end of the respective elongated pin may be substantially flush with a proximal end of the standoff in the intermediate position.

In any implementations, the assembly may include a patient-specific depth stop module releasably securable to the guide body. The depth stop module may include the one or more patient-specific standoffs adjacent the respective one or more passageways.

In any implementations, the guide body may include a first module and a second module releasably securable to each other to capture the position verification member in the respective passageway.

In any implementations, the first module may include the one or more patient-specific contact surfaces.

In any implementations, the at least one position verification member may include a flange. The passageway may include an intermediate section between opposed first and second walls. The flange may be moveable along a length of the intermediate section. The first and second walls may be adapted to abut the flange to limit the starting position and the stopping position. The first module may include the first wall. The second module may include the second wall.

An assembly for a surgical procedure according to an implementation may include a guide including a guide body dimensioned to position a surgical instrument. The guide body may be established by a locating module and a depth stop module. The depth stop module may include one or more standoffs which may be aligned with one or more passageways through the guide body. Each standoff may include a respective patient-specific height. The assembly may include one or more position verification members including a respective indicator and a verification surface adapted to contact tissue. The position verification member may be insertable through the respective passageway to indicate whether the locating module may be positioned on an anatomical surface of a patient in a patient-specific position according to a surgical plan based on a position of the indicator relative to the respective standoff.

In any implementations, the guide may include at least one locating member having a patient-specific surface dimensioned to seat the guide on the anatomical surface in the patient-specific position.

In any implementations, the depth stop module may include at least one patient-specific depth stop dimensioned to limit insertion of a surgical instrument through the guide body.

In any implementations, the guide body may include a guide module including at least one opening adapted to receive a surgical instrument. The guide module may be securable between the locating module and the depth stop module in an assembled configuration. The one or more passageways may extend through the locating module, the guide module and/or the depth stop module in the assembled configuration.

In any implementations, the indicator may be established along a proximal end of the position verification member. The proximal end may be substantially flush with a proximal surface of the respective standoff in response to positioning at least an adjacent portion of the guide on the anatomical surface in the patient-specific position.

In any implementations, a spring member may be configured to bias the respective position verification member distally from the respective passageway.

In any implementations, the one or more position verification members may be releasably securable to the surgical instrument.

A surgical kit according to an implementation may include a surgical instrument including one or more passageways. One or more verification members may include a respective elongated pin and a respective carrier. The elongated pin may have a contact surface and an indicator. The carrier may have a carrier passage dimensioned to receive the respective elongated pin. The elongated pin may be moveable relative to the carrier. The carrier may be adapted to engage the surgical instrument to position the elongated pin in the respective passageway to determine a placement of the surgical instrument on an anatomical surface of a patient based on a position of the indicator.

In any implementations, the one or more verification members may include a respective spring member dimensioned to bias the elongated pin distally from the surgical instrument.

In any implementations, the carrier may include at least one viewing aperture along a periphery of the carrier passage. The indicator may be at least partially alignable with the at least one viewing aperture to indicate the position of the contact surface.

In any implementations, the elongated pin may be moveable in a plurality of positions relative to the carrier. The positions may include a starting position, a stopping position and an intermediate position. The indicator may be alignable with the at least one viewing aperture in the intermediate position but not in the starting position and/or the stopping position.

A surgical kit according to an implementation may include a surgical instrument and one or more position verification members having a respective patient-specific dimension according to a surgical plan. The one or more position verification members may be insertable through respective passageways of the surgical instrument to determine a placement of the surgical instrument on an anatomical surface of a patient.

In any implementations, the position verification members may have different patient-specific lengths associated with a contour of the anatomical surface.

In any implementations, the position verification members may be fixedly secured to each other such that the position verification members may be simultaneously positionable in the respective passageways of the surgical instrument.

In any implementations, the one or more position verification members may include a first alignment feature that may be alignable with a second alignment feature of the surgical instrument to indicate the placement of the surgical instrument on the anatomical surface.

In any implementations, the first alignment feature may be established by a proximal end of the respective position verification member. The second alignment feature may be established along a face of the surgical instrument.

In any implementations, the surgical instrument may include one or more patient-specific contact surfaces dimensioned to seat the surgical instrument in a patient-specific position on the anatomical surface according to the surgical plan.

In any implementations, the one or more passageways may include a first passageway having an opening along a respective one of the patient-specific contact surfaces.

A method of performing an orthopaedic procedure may include placing a surgical instrument on an anatomical surface of a patient. The method may include positioning at least one position verification member through a respective passageway of the surgical instrument such that the at least one position verification member may extend distally from the passageway to contact the anatomical surface. The at least one position verification member may include an indicator moveable in a plurality of positions. The positions may include a starting position, a stopping position and an intermediate position between the starting and stopping positions. The method may include determining that the surgical instrument is placed in a patient-specific position specified by a surgical plan for the patient in response to the indicator being aligned with an alignment feature in the intermediate position, but determining that a placement of the surgical instrument deviates from the patient-specific position in response to the indicator being misaligned with the alignment feature of the surgical instrument.

In any implementations, the surgical instrument may include an aperture. The method may include guiding a surgical tool through the aperture to engage the anatomical surface subsequent to the determining step.

In any implementations, the surgical instrument may include one or more patient-specific locating members having respective contact surfaces dimensioned to place the surgical instrument on the anatomical surface in the patient-specific position. The placing step may include establishing contact between the one or more patient-specific locating members and the anatomical surface.

In any implementations, the contact surface of at least one of the patient-specific locating members may include a first contact surface dimensioned to follow a contour of the anatomical surface.

In any implementations, the first contact surface may include an opening to the respective passageway.

In any implementations, the at least one position verification member may protrude from a proximal end of the passageway in response to the placement of the surgical instrument deviating from the surgical plan.

In any implementations, the method may include biasing the at least one position verification member distally towards the anatomical surface.

A method of performing an orthopaedic procedure according to an implementation may include placing a surgical instrument on an anatomical surface of a patient. The method may include positioning at least one patient-specific position verification members relative to the surgical instrument to contact the anatomical surface. The method may include determining whether the surgical instrument may be placed in a patient-specific position specified by a surgical plan for the patient based on a position of the at least one position verification member relative to the surgical instrument.

In any implementations, the at least one position verification member may include a respective patient-specific length based on the surgical plan.

In any implementations, the at least one position verification member may include a set of position verification members insertable through respective passageways of the surgical instrument. The position verification members may have different patient-specific lengths based on a contour of the anatomical surface.

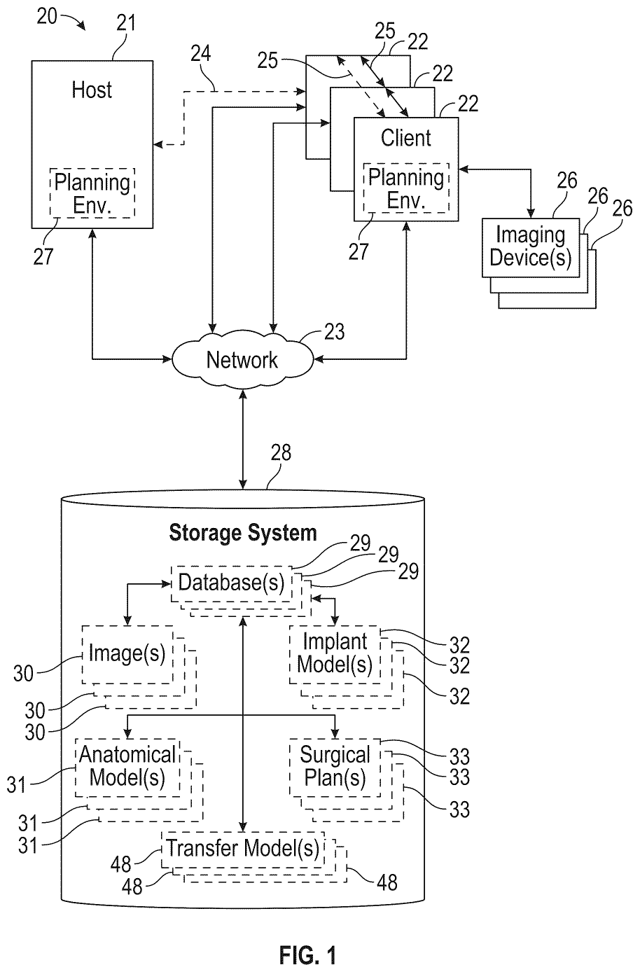

FIG. 1 discloses a surgical planning system 20 according to an implementation. The system 20 may be utilized for planning orthopaedic and/or other surgical procedures, including pre-operatively, intra-operatively and/or post-operatively to create, edit, execute and/or review surgical plans. The system 20 may be utilized for various orthopaedic and other surgical procedures, such as an arthroplasty to repair a joint. The system 20 may be utilized in the design and/or placement of implant(s) and/or surgical instruments (e.g., transfer guides), such as an implant incorporated into an ankle prosthesis or a transfer guide for positioning one or more surgical instruments and/or implants. Although the planning systems and methods disclosed herein primarily refer to repair of a tibia and/or talus during ankle reconstruction, it should be understood that the planning system 20 may be utilized in the repair of other locations of the anatomy and other surgical procedures including repair of other bones and joints such as the shoulder, wrist, hand, hip or knee.

The system 20 may include a host computer 21 and one or more client computers 22. The host computer 21 may be configured to execute one or more software programs. In implementations, the host computer 21 may be more than one computer jointly configured to process software instructions serially or in parallel.

The host computer 21 may be in communication with one or more networks such as a network 23 comprised of one or more computing devices. The network 23 may be a private local area network (LAN), a private wide area network (WAN), the Internet, or a mesh network.

The host computer 21 and each client computer 22 may include one or more computer processors, memory, storage means, network devices, and input and/or output devices and/or interfaces. The input devices may include keyboards, mice and touch screens. The output devices may include monitors, speakers and printers. The memory may include UVPROM, EEPROM, FLASH, RAM, ROM, DVD, CD, a hard drive, or other computer readable medium which may store data and/or other information relating to the planning and implementation techniques disclosed herein. The host computer 21 and each client computer 22 may be a desktop computer, laptop computer, smart phone, tablet, or any other computing device. The interface may facilitate communication with the other systems and/or components of the network 23.

Each client computer 22 may be configured to communicate with the host computer 21 directly via a direct client interface 24 or over the network 23. In another implementation, the client computers 22 may be configured to communicate with each other directly via a peer-to-peer interface 25.

The system 20 may include, or may be coupled to, one or more imaging devices 26. Each client computer 22 may be coupled to one or more imaging devices 26. Each imaging device 26 may be configured to capture or acquire one or more images 30 of patient anatomy residing within a scan field (e.g., window) of the imaging device 26. The imaging device 26 may be configured to capture or acquire two-dimensional (2D) and/or three-dimensional (3D) greyscale and/or color images 30. Various imaging devices 26 may be utilized, such as an X-ray machine, computerized tomography (CT) machine or magnetic resonance imaging (MRI) machine that may obtain one or more images of a patient.

The client computers 22 may be configured to execute one or more software programs, including various surgical tools. Each client computer 22 may be operable to access and locally and/or remotely execute a planning environment 27. The planning environment 27 may be a standalone software package or may be incorporated into another surgical tool. The planning environment 27 may be configured to communicate with the host computer 21 either over the network 23 or directly through the direct client interface 24.

The planning environment 27 may be configured to interact with one or more of the imaging devices 26 to capture or acquire images 30 of patient anatomy. The planning environment 27 may provide a display or visualization of one or more images 30, virtual anatomical (e.g., bone) models 31, virtual implant models 32 and/or virtual surgical transfer (e.g., instrument or guide) models 48 via one or more graphical user interfaces (GUI). The anatomical model 31 may be representative of one or more bones and/or soft tissue. Each image 30, anatomical model 31, implant model 32, transfer model 48 and other data and information may be stored in one or more files or records according to a specified data structure. The implant model 32 may include one or more components. The implant model 32 may be associated with various implants, such as bases (e.g., trays or base plates) configured to be coupled to a respective articulation member, and bone plates configured to interconnect adjacent bones or bone fragments. The articulation member may have an articular surface dimensioned to mate with an articular surface of an opposed bone or implant.

The system 20 may include at least one storage system 28, which may be operable to store or otherwise provide data to other computing devices. The storage system 28 may be a storage area network device (SAN) configured to communicate with the host computer 21 and/or the client computers 22 over the network 23. In implementations, the storage system 28 may be incorporated within, or may be directly coupled to, the host computer 21 and/or client computers 22. The storage system 28 may be configured to store various information, such as one or more of computer software instructions, data, database files and configurations.

In implementations, the system 20 may be a client-server architecture configured to execute computer software on the host computer 21, which may be accessible by the client computers 22 using either a thin client application or a web browser executing on the client computers 22. The host computer 21 may be operable to load the computer software instructions from local storage, or from the storage system 28, into memory and may execute the computer software using the one or more computer processors.

The system 20 may include one or more databases 29. The databases 29 may be stored at a central location, such as the storage system 28. In other implementations, one or more databases 29 may be stored at the host computer 21 and/or may be a distributed database provided by one or more of the client computers 22. Each database 29 may be a relational database configured to associate one or more images 30, anatomical models 31, implant models 32 and/or transfer models 48 to each other and/or respective surgical plan(s) 33. Each surgical plan 33 may be associated with the anatomy of a respective patient. Each image 30, anatomical model 31, implant model 32, transfer model 48 and/or surgical plan 33 may be assigned a unique identifier or database entry. The database 29 may be configured to store data and other information corresponding to the images 30, anatomical models 31, implant models 32, transfer models 48 and/or surgical plans 33 in one or more database records or entries, and/or may be configured to link or otherwise associate one or more files corresponding to each respective image 30, anatomical model 31, implant model 32, transfer model 48 and/or surgical plan 33. Images 30, anatomical models 31, implant models 32, transfer models 48 and/or associated surgical plans 33 stored in the database(s) 29 may correspond to respective patient anatomies from prior, planned and/or hypothetical surgical cases, and may be arranged into one or more predefined categories such as sex, age, ethnicity, defect category, procedure type, surgeon, and/or facility or organization.

Each image 30 and/or anatomical model 31 may include data and other information obtained from one or more medical devices or tools, such as the imaging devices 26. The anatomical model 31 may include coordinate information relating to an anatomy of the patient obtained or derived from image(s) 30 captured or otherwise obtained by the imaging device(s) 26. Each implant model 32 and transfer model 48 may include geometry and/or coordinate information associated with a predefined design or a design established or modified by the planning environment 27. The planning environment 27 may incorporate and/or interface with one or more modeling packages, such as a computer aided design (CAD) package, to render the models 31, 32, 48 as 2D and/or 3D volumes or constructs, which may overlay one or more of the images 30 in a display screen of a GUI.

The implant models 32 may correspond to (e.g., physical) implants and components of various configurations, shapes, sizes, procedures and/or instrumentation. The implant model 32 may be associated with a patient-specific implant for treating a single patient or may be non-patient specific (e.g., generic) for treating different patients. Each implant may include, or may otherwise be associated with, one or more components that may be situated at a surgical site including grafts and various fixation devices such as screws, anchors, nails and suture. Each implant model 32 may correspond to a single component or may include two or more components that may be configured to establish an assembly. The implant model 32 may include a base (e.g., base plate or tray) coupled to an articulation member, bone plates configured to interconnect adjacent bones or bone fragments, intermedullary nails, suture anchors, etc. The articulation member may have an articular surface dimensioned to mate with an articular surface of an opposed bone or implant. Each implant and associated component(s) may be formed of various materials, including metallic and/or non-metallic materials. Each bone model 31, implant model 32 and transfer model 48 may correspond to 2D and/or 3D geometry and may be utilized to generate a wireframe, mesh and/or solid construct in a display.

Each surgical plan 33 may be associated with one or more of the images 30, bone models 31, implant models 32 and/or transfer models 48. The surgical plan 33 may include various parameters associated with the images 30, bone models 31, implant models 32 and/or transfer models 48. The surgical plan 33 may include parameters relating to bone density and bone quality associated with patient anatomy captured in the image(s) 30. The surgical plan 33 may include parameters including spatial information relating to relative positioning and coordinate information of the selected bone model(s) 31, implant model(s) 32 and/or transfer model(s) 48.

The surgical plan 33 may include one or more revisions to a bone model 31 and information relating to a position of an implant model 32 and/or transfer model 48 relative to the original and/or revised bone model 31. The surgical plan 33 may include coordinate information relating to the revised bone model 31 and a relative position of the implant model 32 and/or transfer model 48 in predefined data structure(s). The planning environment 27 may be configured to make one or more revisions to a transfer model 48 automatically or in response to user interaction with the user interface. Revisions to each bone model 31, implant model 32, transfer model 48 and/or surgical plan 33 may be stored in the database 29 automatically and/or in response to user interaction with the system 20.

One or more surgeons and other users may be provided with a planning environment 27 via the client computers 22 and may simultaneously access each image 30, bone model 31, implant model 32, transfer model 48 and/or surgical plan 33 stored in the database(s) 29. Each user may interact with the planning environment 27 to create, view and/or modify various aspects of the surgical plan 33. Each client computer 22 may be configured to store local instances of the images 30, bone models 31, implant models 32, transfer models 48 and/or surgical plans 33, which may be synchronized in real-time or periodically with the database(s) 29. The planning environment 27 may be a standalone software package executed on a client computer 22 or may be provided as one or more services executed on the host computer 21.

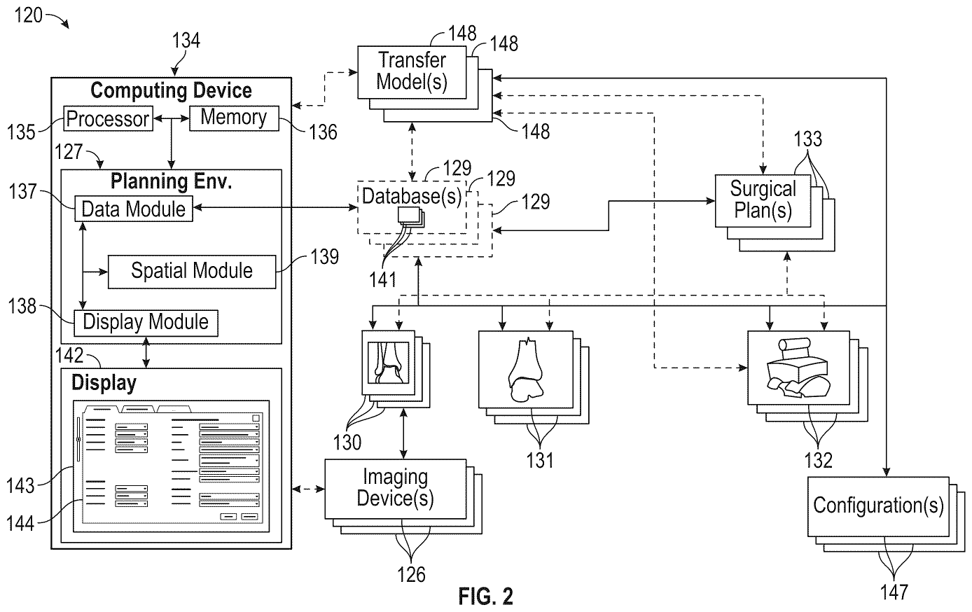

FIG. 2 discloses a planning system 120 for planning a surgical procedure. In this disclosure, like reference numerals designate like elements where appropriate and reference numerals with the addition of one-hundred or multiples thereof designate modified elements that are understood to incorporate the same features and benefits of the corresponding original elements. The system 120 may be incorporate any of the features of system 20 and/or vice versa. The system 120 may be utilized to plan and implement various orthopaedic and other surgical procedures, such as an arthroplasty to repair various bones and/or joints. The system 120 may be utilized in planning the preparation of one or more bones. The system 120 may be utilized in planning placement of implant(s) to restore functionality to the joint. Although the planning systems and methods disclosed herein primarily refer to repair of an ankle, it should be understood that the planning system 120 may be utilized in the repair of other anatomy of the patient and other surgical procedures including repair of other joints such as a shoulder, wrist, hand, hip or knee.

The system 120 may include a computing device 134 including one or more processors 135 coupled to memory 136. The computing device 134 may include any of the computing devices disclosed herein, including the host computer 21 and/or client computer 22. The processor(s) 135 may be configured to collectively execute a planning environment 127 for creating, editing, executing and/or reviewing one or more surgical plans 133 and any associated anatomical (e.g., bone) models 131, implant models 132 and/or transfer models 148 during pre-operative, intra-operative and/or post-operative phases of a surgery.

The planning environment 127 may include a data (e.g., interface) module 137, a display module 138 and a spatial (e.g., evaluation) module 139. Although three modules are disclosed, it should be understood that fewer or more than three modules may be utilized and/or one or more of the modules may be combined to provide the disclosed functionality.

The data module 137 may be configured to access, retrieve and/or store data and other information in the database(s) 129 corresponding to one or more images 130 of patient anatomy, anatomical model(s) 131, implant model(s) 132, transfer model(s) 148 and/or surgical plan(s) 133. The data and other information may be stored in one or more databases 129 as one or more records or entries 141. In implementations, the data and other information may be stored in one or more files that may be accessible by referencing one or more objects or memory locations referenced by the records 141.

The memory 136 may be configured to access, load, edit and/or store instances of one or more images 130, anatomical models 131, implant models 132, transfer models 148 and/or surgical plans 133 in response to one or more commands from the data module 137. The data module 137 may be configured to access a virtual anatomical model 131 from memory, such as the memory 36 and/or storage system 128. The anatomical model 131 may be associated with bone(s) and/or joint(s) of a patient. The data module 137 may be configured to cause the memory 136 to store a local instance of the image(s) 130, bone model(s) 131, implant model(s) 132, transfer model(s) 148 and/or surgical plan(s) 133, which may be synchronized with the records 141 in the database(s) 129.

The data module 137 may be configured to receive data and other information corresponding to at least one or more images 130 of patient anatomy from various sources such as the imaging device(s) 126. The data module 137 may be configured to command the imaging device 126 to capture or otherwise acquire the images 130 automatically and/or in response to user interaction.

The display module 138 may be configured to display data and other information relating to one or more surgical plans 133 in at least one graphical user interface (GUI) 143, including one or more of the images 130, anatomical models 131, implant models 132 and/or transfer models 148. The computing device 134 may incorporate, or may be coupled to, a display device 142. The user interface 143 may include one or more display windows 144. The display module 138 may be configured to cause the display device 142 to display information in the display window(s) 144 and/or another portion of the user interface 143. A surgeon or other user may interact with the user interface 143 via the planning environment 127 to view one or more images 130 of patient anatomy and/or any associated anatomical models 131, implant models 132 and/or transfer models 148. The surgeon or other user may interact with the user interface 143 via the planning environment 127 to create, edit, execute and/or review one or more surgical plans 133.

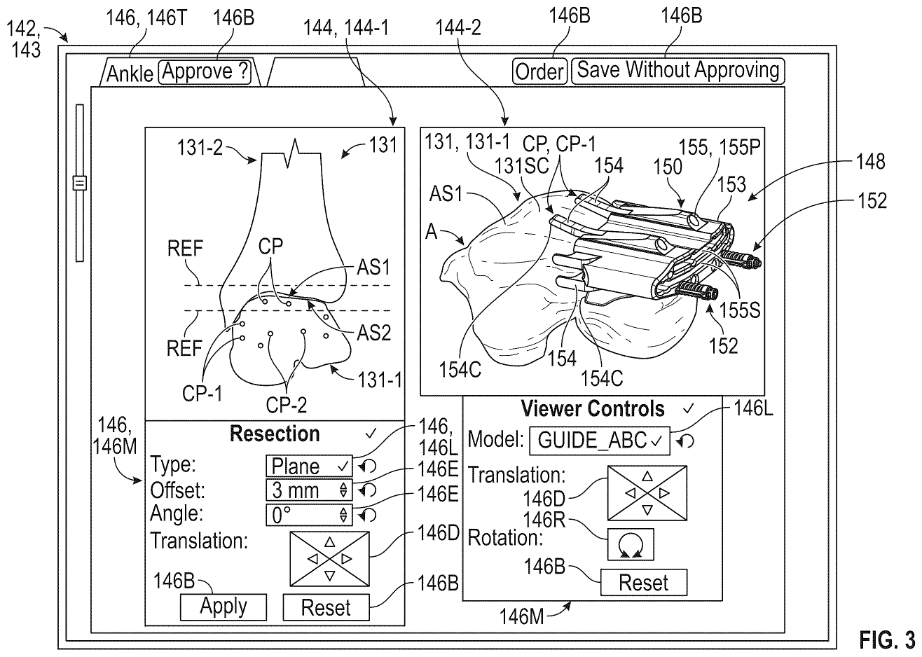

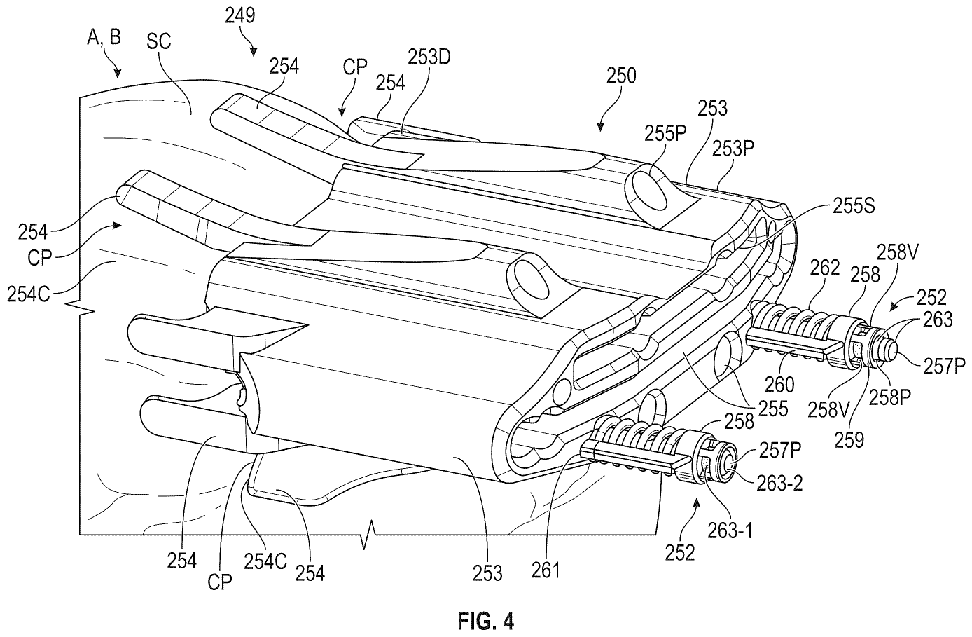

The planning system 120 may be configured to access, generate, review, edit and/or approve one or more configurations 147 associated with respective physical implant(s) and/or surgical instrument(s) (e.g., transfer guides), including any of the implants and instruments disclosed herein. The virtual implant model 132 may be representative of one or more physical implants, which may be associated with a prosthesis. The virtual transfer model 148 (e.g., FIG. 3) may be representative of a respective physical surgical instrument, such as a physical transfer guide and/or one or more associated position verification members (e.g., FIG. 4). The configuration 147 may specify various information for forming and/or configuring an instance of a respective physical implant, transfer guide and/or position verification member(s), which may be based on a respective virtual implant model 132 or transfer model 148. In implementations, the configuration 147 may specify a geometry and/or arrangement of one or more position verification members relative to the associated transfer guide (e.g., FIG. 4). Each configuration 147 may include one or more files in a predetermined data structure or format. In implementations, the configuration 147 may include a coordinate set and/or other information such as material selection(s) associated with volume(s) of the physical implant, transfer guide and/or transfer member(s). The physical implant(s), transfer guide(s) and/or transfer member(s) may be formed utilizing various techniques, including any of the techniques disclosed herein such as rapid prototyping (e.g., printing) and other additive manufacturing techniques, molding, casting and/or machining.

Referring to FIG. 3, with continuing reference to FIGS. 1-2, the user interface 143 may include one or more display windows 144 and one or more objects 146. The objects 146 may include graphics such as menus, tabs and buttons accessible by user interaction, such as tabs 146T, buttons 146B, drop-down lists 146L, menus 146M, (e.g., text) entry fields 146E, directional indicators 146D, 146R and graphics. Geometric objects including selected bone model(s) 131, implant model(s) 132 and/or transfer model(s) 148 and/or other information relating to the surgical plan 133 may be displayed in one or more of the display windows 144.

The display module 138 may be configured to display one or more selected anatomical models 131, implant models 132 and/or transfer models 148 in the display windows 144. The display module 138 may be configured such that the selected anatomical model(s) 131, implant model(s) 132 and/or transfer model(s) 148 may be selectively displayed and hidden (e.g., toggled) in one or more of the display windows 144 in response to user interaction with the user interface 143, which may provide the surgeon with enhanced flexibility in reviewing aspects of the surgical plan 133.

The data module 137 may be configured to access one or more anatomical models 131 including a first bone model 131-1 and/or second bone model 131-2 from the database 129, which may occur automatically or in response to user interaction with the user interface 143. The data module 137 may be configured to store an instance of the first bone model 131-1 and second bone model 131-1 in the memory 136. The first bone model 131-1 and second bone model 131-2 may be associated with a joint. One of the bone models 131 may be associated with a long bone, and another one of the bone models 131 may be associated with an adjacent bone that may cooperate to establish an ankle or another joint of a patient. In the implementation of FIG. 3, the first bone model 131-1 may be associated with a talus, and the second bone model 131-2 may be associated with a tibia. The display module 138 may be configured to display the bone models 131-1, 131-2 in at least one of the display windows 144 of the user interface 143.

The user interface 143 may be configured with any number of display windows 144 in accordance with the teachings disclosed herein. The display windows 144 may include a first display window 144-1 and/or a second display window 144-2. The display windows 144-1, 144-2 may be configured to display a 2D and/or 3D representation of the selected anatomical model(s) 131, including the first and/or second bone models 131-1, 131-2.

The first display window 144-1 may be configured to display the first bone model 131-1 and second bone model 131-2 and/or associated articular surfaces AS1, AS2 relative to each other. The spatial module 139 may be configured to position the bone models 131-1, 131-2 into contact with each other at a specified or defined position and orientation, which may be according to user interaction with the window 144-1, menu 146M, and/or other objects 146 of the user interface 143.

The second display window 144-2 may be configured to display a transfer model 148 relative to one or more of the bone model(s) 131. The surgeon or clinical user may interact with the display windows 144-1, 144-2, the directional indicators 146D, 146R, and/or another portion of the user interface 143 to move the selected bone model 131 and/or selected transfer model 148 in 2D space (e.g., up, down, left, right) and/or 3D space (e.g., rotation, tilt, zoom).

The transfer model 148 may include, or may otherwise be associated with, at least one instrument (e.g., guide) model 150 and/or one or more position verification member models 152. The spatial module 139 may be operable to position the guide model 150 and/or associated verification member model(s) 152 relative to each other and/or the anatomical model 131. The guide model 150 may include a guide (e.g., main) body 153. One or more locating members 154 may extend from the guide body 153.

The surgeon or clinical user may interact with the menu 146M, directly with the display window 144-2 using a pointer and/or with another portion of the user interface 143 to set (e.g., select) one or more parameters (e.g., settings) associated with the transfer model 148. In implementations, the user may select a total number of the locating members 154 and/or a position of the locating member(s) 154 relative to the guide body 153 of the guide model 150 and/or anatomy A of the patient.

The guide model 150 and/or associated verification member model(s) 152 may be positioned along the anatomical model 131 at one or more respective contact points CP (e.g., CP-1, CP-2). The contact points CP may associated with one or more landmarks of the anatomical model 131. Various techniques may be utilized to specify the contact points CP. In implementations, the spatial module 139 may be configured to automatically position the guide model 150 relative to the anatomical model 131 based on the associated trajectories associated with planned alterations to the anatomy. The surgeon or clinical user may interact with the user interface 143 to specify contact point(s) CP associated with the respective locating member(s) 154 and/or other portions of the guide model 150 along or otherwise relative to the anatomical model 131, including along a 2D and/or 3D surface contour 131SC of the anatomy A.

The guide model 150 may be patient-specific such that the guide model 150 may fit the anatomical model 131 in a single, patient-specific position and orientation to establish the contact point(s) CP-1. The guide model 150 may be configured to position the verification member model(s) 152 such that the verification member model(s) 152 may establish contact with the anatomical model 131 at the respective contact point(s) CP-2 based on the patient-specific position and orientation of the guide model 150 relative to the anatomical model 131. The guide model 150 may be positioned on the bone model 131 to establish a trajectory of one or more guide apertures 155 (e.g., slots or holes). The guide apertures 155 may have various geometries, such as a substantially elliptical (e.g., circular) cross section or a slot. For the purposes of this disclosure, the term “substantially” means±10 percent of the stated value or relationship unless otherwise indicated. The guide apertures 155 may include one or more resection slots 155S and/or passages (e.g., holes) 155P. The resection slot 155S may be associated with a reference (e.g., resection) plane REF. The surgeon or clinical user may interact with the display window(s) 144 and/or another portion of the user interface 143 to adjust or otherwise set the reference plane(s) REF and/or trajectories.

The guide body 153 may be positioned at a patient-specific placement (e.g., position and/or orientation) relative to the anatomical model 131. The spatial module 139 may be operable to extrude the locating member(s) 154 from the guide body 153 to the respective contact points CP.

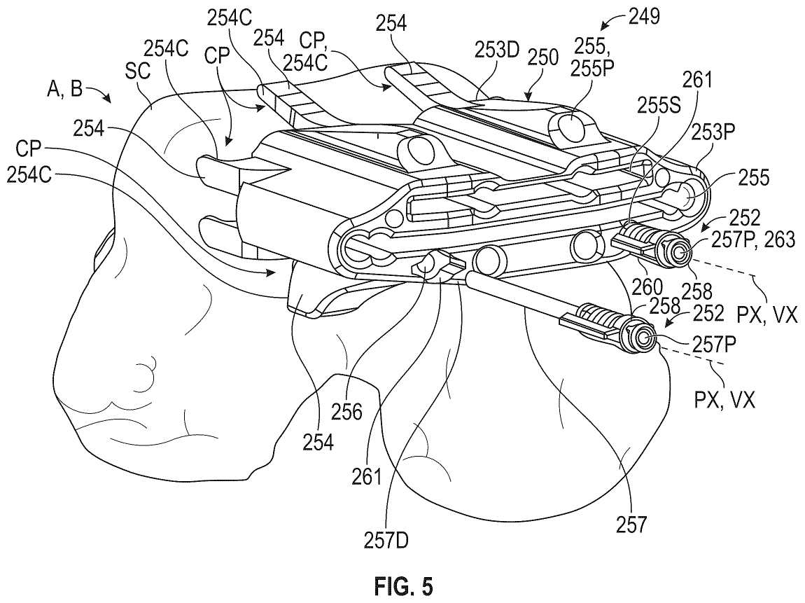

The locating members 154 may have at least one patient-specific dimension (e.g., length). The contact surface(s) 154C of the locating member(s) 154 may be geometric (e.g., flat, curved or rounded). The contact surfaces of the locating members 154 and/or other portions of the transfer model 148 may be patient-specific and may be dimensioned to follow a surface contour of a bone associated with the anatomical model 131, such as an articular surface and/or a non-articular surface of the bone of a patient. In implementations, one or more of the locating members 154 may have patient-specific contact surface(s) 154C which may be contiguous and/or non-contiguous with each other. The contact surface 154C may be dimensioned to follow the surface contour 131SC of the bone model 131. The surface contour 131SC may be associated with a surface contour SC of a respective bone B of the patient (e.g., FIGS. 4-5). The surgeon or clinical user may interact with a button 146B and/or another portion of the user interface 143 to approve the setting(s) associated with the transfer model 148.

Referring to FIGS. 4-5, with continuing reference to FIGS. 2-3, a system (e.g., assembly or kit) 249 for a surgical procedure according to an implementation is disclosed. The system 249 may be utilized to perform any of the techniques disclosed herein. The system 249 may be associated with a respective transfer model 148 (e.g., FIG. 3). The system 249 may include a surgical instrument (e.g., transfer guide) 250 and/or one or more position verification members 252. The guide 250 and verification member(s) 252 may be included in a surgical kit. The guide 250 and verification member(s) 252 may be arranged to establish an assembly, including preoperatively and/or intraoperatively. The transfer guide 250 and verification members 252 may incorporate any of the features disclosed herein, including the features of the transfer model 148. The guide 250 may be utilized to position one or more surgical devices relative to anatomy such as instrumentation and/or implant(s) of a prosthesis. The anatomy may include one or more bones and/or joints of a patient. The bones may include one or more bones associated with a joint, including various long bones such as a humerus, femur, or tibia. The joints may include a shoulder, ankle, knee, hip or elbow joint. The verification members 252 may be arranged relative to the guide 250 to indicate a degree of fit between the contact surface(s) of the guide 250 and an anatomical surface of the anatomy, which may be specified in a surgical plan 133 (e.g., FIG. 2). Two or more verification members 252 may be arranged at different positions relative to the guide 250 to check the degree of fit at different positions relative to the anatomy. The verification members 252 may be utilized to perform other functions, such as checking a depth of a hole drilled into a bone.

The transfer guide 250 may include a guide (e.g., main body) body 253. The guide body 253 may be dimensioned to guide one or more surgical devices (e.g., instruments). The surgical devices may include cutting instrument and/or guide elements. Various cutting instruments may include a drill, reamer or saw blade suitable for removing bone or other tissue. Various guide elements may be utilized, such as a Kirshner (K-wire) insertable in bone. The guide body 253 may include one or more guide apertures 255. The guide apertures 255 may include one or more resection slots 255S and/or passages (e.g., holes) 255P. The guide 250 may be seated on a bone B of the anatomy A to set the trajectory of the respective guide aperture(s) 255, which may be specified in a surgical (e.g., preoperative) plan 133 for the patient.

The guide 250 may be adapted to contact an anatomical surface of the anatomy A based on the surgical plan 133. Various techniques may be utilized to position the guide 250 according to the surgical plan 133. The guide body 253 may extend between a first (e.g., proximal) end 253P and a second (e.g., distal) end 253D. The guide 250 may include one or more locating members 254. The locating members 254 may extend from the distal end 254D and/or another portion of the guide body 253. The locating members 254 and/or another portion of the guide 250 may include one or more patient-specific dimensions and/or patient-specific surfaces to establish a pre-selected position and orientation of the guide 250 relative to an anatomical surface of the anatomy A. The guide body 253 and/or another portion of the guide 250 may include one or more patient-specific contact surfaces 254C. In implementations, the patient-specific contact surface 254C may be established by a respective one of the locating members 254. The locating members 254 may include respective contact surfaces 254C, which may be dimensioned according to any of the techniques disclosed herein. The locating members 254 may be dimensioned to seat the guide 250 on a surface contour SC of the bone B to establish the trajectories of the respective guide aperture(s) 255. In other implementations, the locating member(s) 254 and/or another portion of the alignment guide 250 may be configurable to establish the position and orientation of the guide 250 relative to the anatomy A based on one or more settings, which may be specified in the surgical plan 133.

The verification member(s) 252 may be operable to verify a placement (e.g., seating) of the guide 250 on the anatomy A according to the surgical plan 133. The guide body 253 may include one or more passageways 256. Each passageway 256 may extend along respective passage axes PX. The passage axes PX may be substantially parallel to each other or may be transverse. Each passageway 256 may be adapted to receive a respective verification member 252. Each verification member 252 may be receivable in a respective one of the passageways 256. The verification members 252 may be the same or may differ from each other. The verification members 252 may be independently movable relative to the guide 250. In implementations, the guide aperture(s) 255 may include the passageway(s) 256. The passageway(s) 256 may be utilized to position one or more surgical instruments, such as one or more guide elements and/or drills.

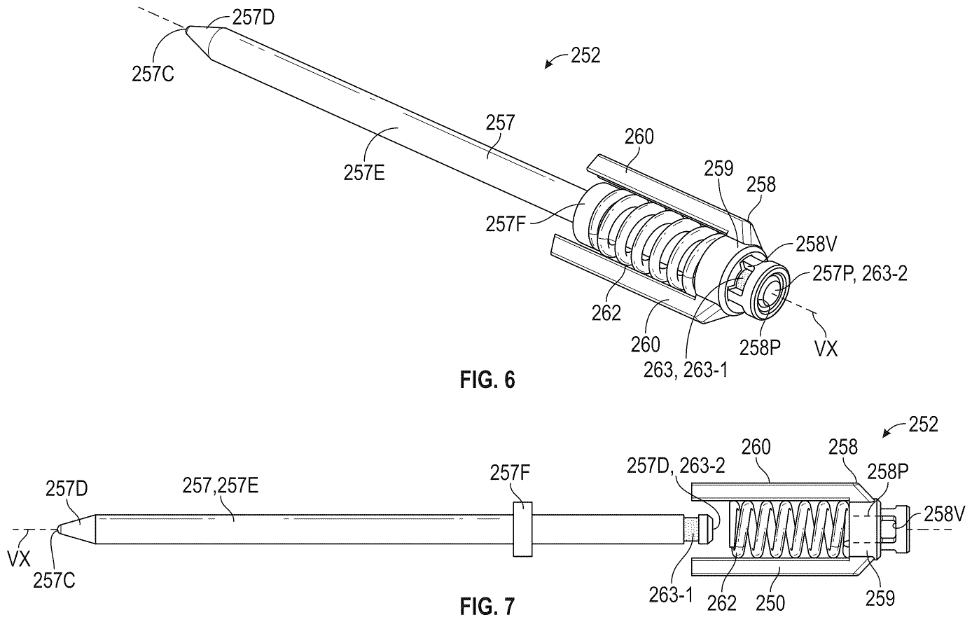

Referring to FIGS. 6-7, with continuing reference to FIGS. 4-5, an implementation of a verification member 252 is disclosed. The verification member 252 may include a main (e.g., indicator) body 257. The indicator body 257 may have various geometries. The indicator body 257 may be patient-generic. In other implementations, the indicator body 257 may be patient-specific and may have one or more patient-specific dimensions. In implementations, the indicator body 257 may be an elongated pin. The indicator body 257 may extend along a verification axis VX of the verification member 252 between a first (e.g., proximal) end 257P and a second (e.g., distal) end 257D. The proximal end 257P may be established by a head of the indicator body 257. The indicator body 257 may include a contact (e.g., verification) surface 257C adapted to contact tissue at a respective contact point CP along the patient anatomy. The verification surface 257C may be determined based on the surgical plan 133. In implementations, the verification surface 257C may be established adjacent to the distal end 257D. The verification surface 257C may be established at a distalmost (e.g., terminal) position of the indicator body 257. In other implementations, the verification surface 257C may be established along a periphery 257E of the indicator body 257.

The verification member 252 may be monolithic or may include two or more components fixedly attached or otherwise secured to each other. The components may be moveable relative to each other. In implementations, the verification member 252 may include a housing (e.g., carrier) 258. The carrier 258 may be patient-generic. In other implementations, the carrier 258 may be patient-specific and may have one or more patient-specific dimensions. In some implementations, the indicator body 257 may be patient-generic and the carrier 258 may be patient-specific, or vice versa. The carrier 258 may be coupled to the indicator body 257. The indicator body 257 may be moveable relative to the carrier 258. The carrier 258 may have a carrier passage 258P dimensioned to receive the indicator body 257. The carrier passage 258P may extend along the axis VX.

The carrier 258 may include a main (e.g., carrier) body 259 that may establish the passage 258P. The carrier 258 may include one or more extensions 260 that may be extend (e.g., distally) from the carrier body 259. In implementations, the extensions 260 may be distributed about the axis VX. The extensions 260 may be distributed about the periphery 257E of the indicator body 257.

In the implementation of FIGS. 4-5, the extensions 260 and/or another portion of the carrier 258 may be adapted to engage the guide body 253 when the indicator body 257 is inserted through a respective passageway 256 (e.g., FIG. 5). The indicator body 257 may be movable along the passageway 256 to position the indicator body 257 relative to the anatomy A. The guide body 253 may include one or more receptacles (e.g., counterbores) 261 dimensioned to at least partially receive the carrier 258. The carrier 258 may be at least partially receivable in the receptacle 261 to set the position of the indicator body 257 relative to the guide body 253. The receptacle 261 may be dimensioned to interfit with a profile of the carrier 258. The extension(s) 260 may serve as keys and the receptacles 261 may serve as keyways. The extension(s) 260 may mate with a periphery of the receptacle 261 to limit relative rotation between the guide 250 and verification member 252. The receptacle 261 may extend from a first (e.g., proximal) end of the passageway 256. In implementations, the verification member 252 and/or indicator body 257 may be patient-generic (e.g., reusable). The receptacle(s) 261 may have a respective patient-specific dimension (e.g., depth), which may be the same or may differ from each other. The receptacle 261 may be dimensioned to set a position of the carrier 258 and/or indicator body 257 relative to the surface contour SC of the anatomy A based on a surgical plan 133 associated with the patient. The receptacles 261 may have different patient-specific depths associated with contact between the verification members 252 and the respective contact points CP along the anatomy A, which may facilitate reuse of (e.g., patient-generic) verification members 252 for different patients. Dimensioning the receptacle 261 according to the techniques disclosed herein may improve alignment and placement of the verification member 252.

The indicator body 257 may be movable in a plurality of positions relative to the carrier 258. The positions may include a starting (e.g., distal) position, a stopping (e.g., proximal) position and one or more intermediate positions between the starting and stopping positions.

The verification member 252 may include at least one indicator 263. The indicator(s) 263 may be configured to indicate a position of the indicator body 257 relative to the carrier 258, the guide 250 and/or anatomy associated with a respective (e.g., patient-specific) contact point CP. The indicator(s) 263 may be aligned with respective alignment feature(s) to indicate the position of the guide 250 relative to the anatomy. The alignment feature(s) may be established by the carrier 258 and/or the guide 250. The planning environment 127 may be operable to establish a configuration 147 of the guide 250 and/or verification member(s) 252 such that the indicator(s) 263 may be substantially aligned with the respective alignment feature(s) when the guide 250 may be positioned on the anatomy A according to the respective surgical plan 133. The configuration 147 may include dimension(s) and/or geometry of the guide 250 and/or verification member(s) 252, a position and/or dimension(s) of the indicator(s) 263 relative to the respective verification member(s) 252, and/or a position and/or dimension(s) of the respective alignment feature(s).

The indicator 263 may be moveable in a plurality of positions relative to the guide body 253 in response to translation of the respective indicator body 257 along the respective passageway 256. The positions may include a starting (e.g., distal) position, a stopping (e.g., proximal) position and one or more intermediate position between the starting and stopping positions. One of the positions, such as the intermediate position, may be associated with a predetermined placement (e.g., seating) of the patient-specific contact surface(s) 254C of the guide 250 relative to an anatomical surface of the patient. The anatomical surface may be associated with a surface contour SC of the respective bone B.

The indicator 263 may be visually distinct from adjacent portions of the indicator body 257 and/or carrier 258. In implementations, the indicator 263 may have a different geometry, color and/or shading. The indicator 263 may have various geometries, such as a line or band (e.g., hash mark or laser line). The indicator 263 may be associated with various text. The surgeon or clinical user may interpret a position of the indicator 263 to determine whether the guide 250 may be properly seated on the anatomy A according to the surgical plan 133.

The indicator(s) 263 may include a first indicator 263-1 and/or a second indicator 263-2. The first indicator 263-1 may be established adjacent to the proximal end 257P of the indicator body 257. In implementations, the first indicator 263-1 may be established along the periphery 257E of the indicator body 257. The first indicator 263-1 may be spaced apart from the proximal end 257P and/or distal end 257D of the indicator body 257. The second indicator 263-2 may be established along and/or by the proximal end 257P of the indicator body 257. The guide 250 may be formed such that the indicator(s) 263 may indicate a (e.g., correct) positioning of the guide 250 on the anatomy A when the respective verification surface(s) 257C contact the anatomy A at the respective patient-specific contact points CP specified by the surgical plan 133.

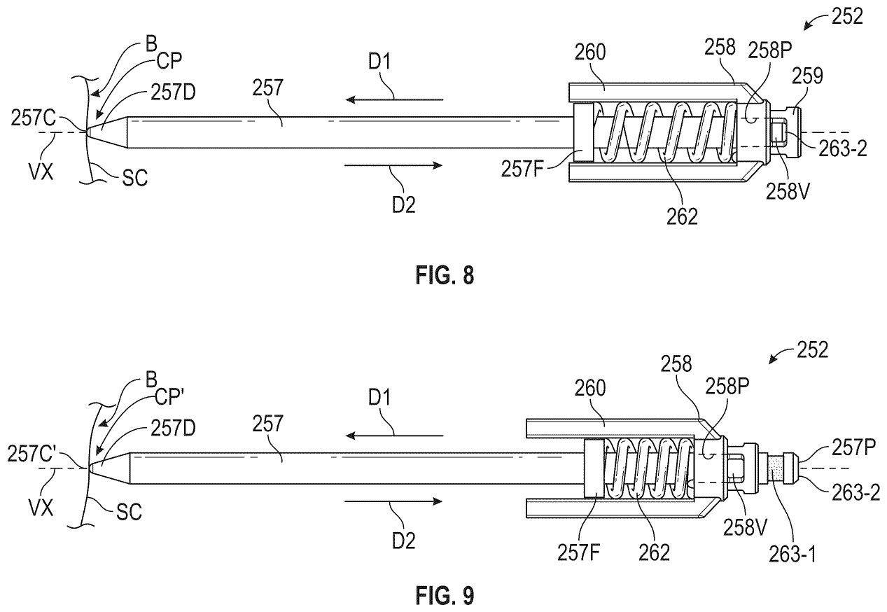

The verification member 252 may be spring-loaded. In implementations, the verification member 252 may include a spring member 262. The spring member 262 may be dimensioned to bias the indicator body 257 and/or another portion of the respective verification member 262 in a first (e.g., distal) direction D1 (e.g., FIGS. 8-9). Various spring members may be utilized, such as a coil, leaf or wave spring. The spring member 262 may be a coil spring that may extend about the periphery 257E of the indicator body 257. The spring member 262 may be attached (e.g., welded) or otherwise secured to the carrier body 259 and/or another portion of the carrier 258. The spring member 262 may be captured between the carrier body 259 and a flange 257F of the respective indicator body 257. The spring member 262 may be dimensioned to bias the indicator body 257 and/or another portion of the respective position verification member 262 toward the starting position. The starting position of the indicator body 257 and/or indicator(s) 263 may be associated with an uncompressed state of the spring member 262. The stopping position may be associated with a compressed state of the spring member 262. The indicator body 257 may be moved in a second (e.g., proximal) direction D2 to at least partially compress the spring member 262 (e.g., FIGS. 8-9). The second direction D2 may be opposed to the first direction D1. In the implementation of FIGS. 8-9, the position of the indicator(s) 263 relative to the carrier 258 may be associated with contact between the contact surface 257C and respective contact points CP, CP′ along the surface contour SC of the bone B.

The proximal end 257P of the indicator body 257 may be situated in the respective passageway 258P in the starting position (e.g., FIG. 8). The proximal end 257P of the indicator body 257 may protrude from the passageway 258P in the stopping position (e.g., FIG. 9). The proximal end 257P of the indicator body 257 may be substantially flush with a rim of the respective passageway 258P in the intermediate position (e.g., FIG. 6). In implementations, the rim of the passageway 258P may be established along a (e.g., proximal face) of the carrier 258, which may serve as an alignment feature for the second indicator 263-2.

The carrier 258 may include at least one opening (e.g., viewing aperture or window) 258V along a periphery of the carrier passage 258P. In implementations, the carrier 258 may include an array of viewing apertures 258V distributed about the periphery of the carrier passage 258P. The first indicator 263-1 may be visually distinct from adjacent portions of the carrier 258 bounding the viewing aperture(s) 258V. In implementations, the viewing aperture(s) 258V may serve as alignment feature(s) for the first indicator 263-1. The first indicator 263-1 may be is at least partially alignable with the viewing aperture(s) 258V to indicate the position of the verification surface 257C (e.g., FIGS. 4 and 6). In implementations, the first indicator 263-1 may be alignable with the viewing aperture(s) 258V in the intermediate position but not in the starting position and/or the stopping position. The indicators 263-1, 263-2 may be visible at different angles relative to the guide 250 and/or carrier 258, which may improve making a determination of the placement when the guide 250 may be situated at a surgical site.

Various techniques may be utilized to establish the guide 250 and verification members 252. The guide 250 and verification members 252 may incorporate various metallic and/or non-metallic materials, such as a surgical grade steel or titanium, and/or non-metallic materials such as a biocompatible polymer. The guide body 253 may be monolithic or may include one or more components fixedly attached or otherwise secured to each other. The guide 250 and verification members 252 may be formed utilizing various techniques, including any of the techniques disclosed herein such as rapid prototyping (e.g., printing) and other additive manufacturing techniques, casting and/or machining.

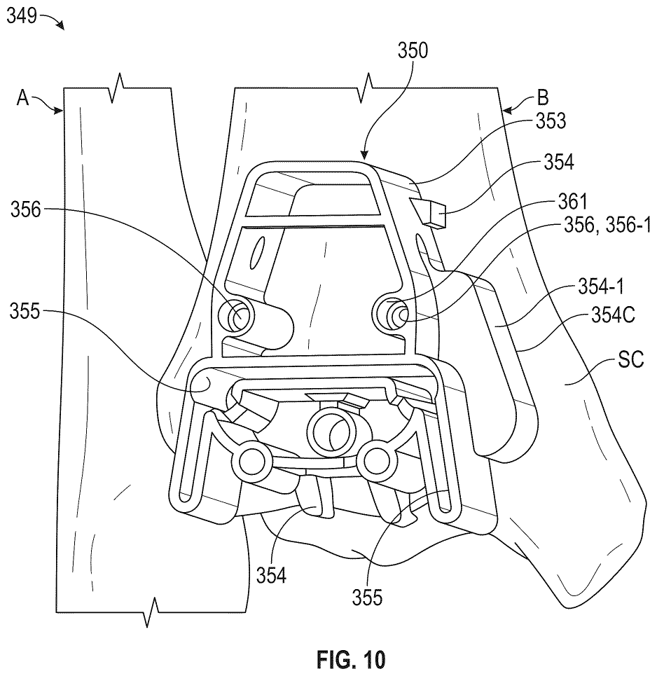

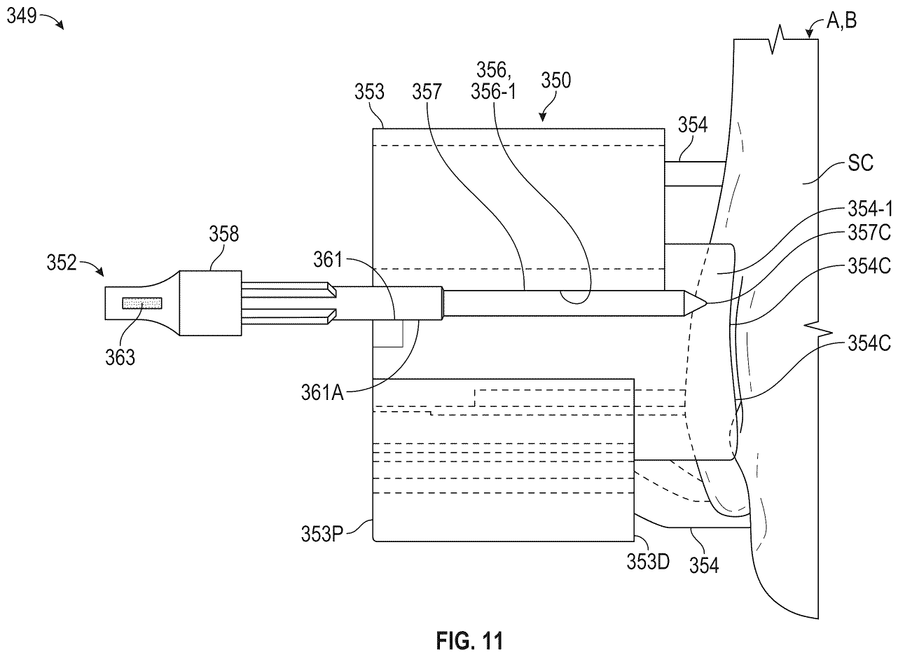

FIGS. 10-11 disclose a surgical system 349 according to another implementation. The system 349 may include a transfer guide 350 and one or more position verification members 352 (FIG. 11). The guide 350 may include a guide body 353 and one or more locating members 354. Each locating member 354 may have one or more patient-specific dimensions and/or patient-specific contact surfaces 354C. In implementations, the locating member(s) 354 may have one or more patient-specific contact surfaces 354C, such as the locating member 354-1. The patient-specific surface 354C of the locating member 354-1 may have a contour that may be a substantial negative of a surface contour SC of a respective surface of an anatomy A, including an articular and/or non-articular surface of a bone B (e.g., tibia).

Referring to FIG. 11, with continuing reference to FIG. 10, the guide body 353 may include one or more passageways 356, such as a first passageway 356-1. The first passageway 356-1 may extend through the locating member 354-1. The first passageway 356-1 may have an opening along the patient-specific contact surface 354C of the locating member 354-1. The guide body 353 may include one or more receptacles 361 at least partially aligned with one or more respective passageways 356, such as the first passageway 356-1.

The verification member 352 may include an indicator body 357. The indicator body 357 may be insertable in the passageway 356. The verification member 352 may include a carrier 358. The carrier 358 may be adapted to abut a (e.g., distal) surface 361A bounding the receptacle 361. The surface 361A may establish a floor of the receptacle 361. The surface 361A may be established at a patient-specific depth relative to the contact surface 354C of the locating member 354, which may be established based on a surgical plan 133 (e.g., FIG. 2). The surface 361A may be dimensioned to abut the carrier 358 to set a position of the carrier 358 relative to the contact surface 354C and/or guide body 353.

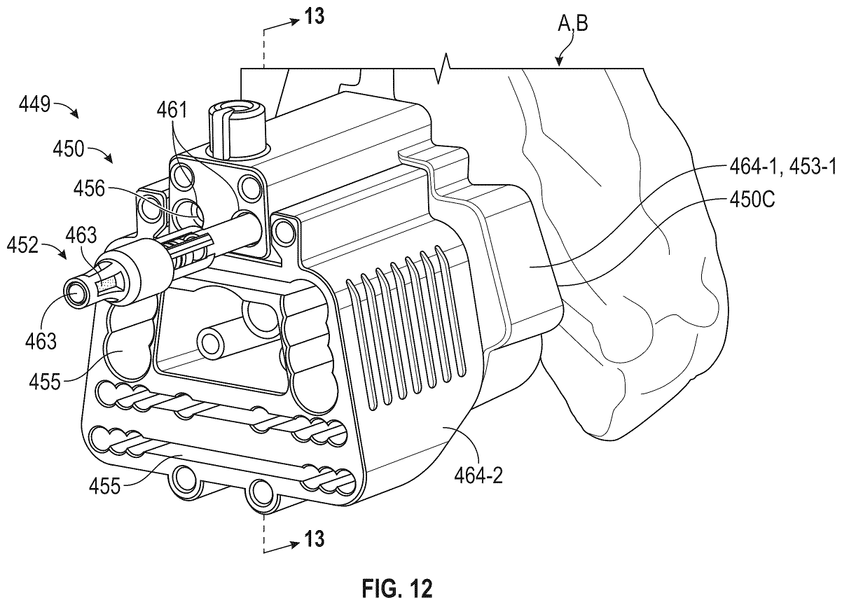

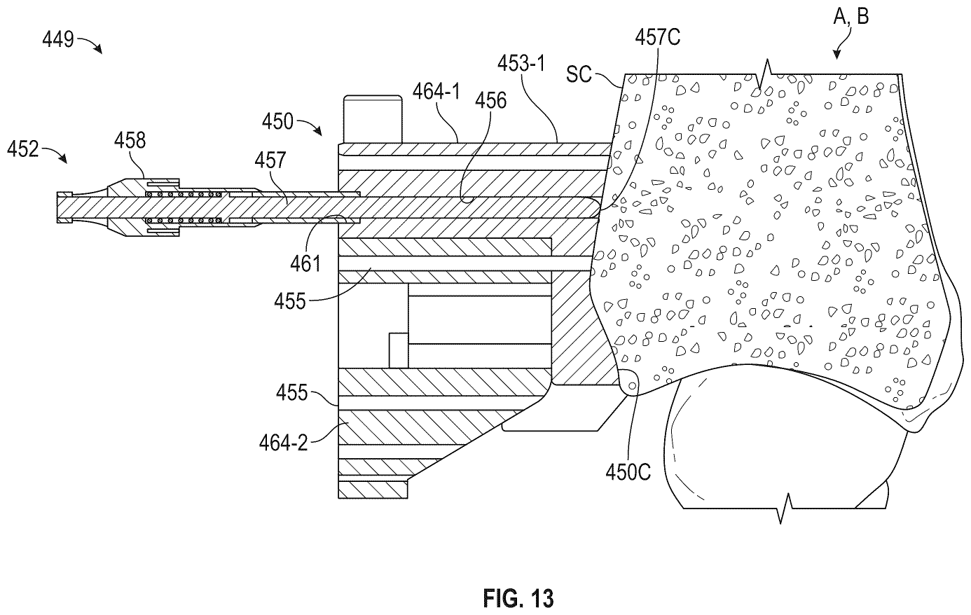

FIGS. 12-13 disclose a surgical system 449 according to another implementation. The system 449 may include a transfer guide (e.g., assembly) 450 and one or more position verification members 452 (e.g., FIG. 11). The transfer guide 450 may include two or more components (e.g., modules) 464, such as a first (e.g., locating or positioning) component 464-1 and/or a second (e.g., guide) component 464-2. The components 464-1, 464-2 may be releasably secured or otherwise coupled to each other. The locating component 464-1 may be adapted to carry the guide component 464-2. In implementations, the locating component 464-1 may be patient-specific and/or the guide component 464-2 may be patient-generic (e.g., reusable). A guide body 453-1 of the locating component 464-1 may have one or more patient-specific contact surfaces 450C, which may be dimensioned according to any of the techniques disclosed herein.

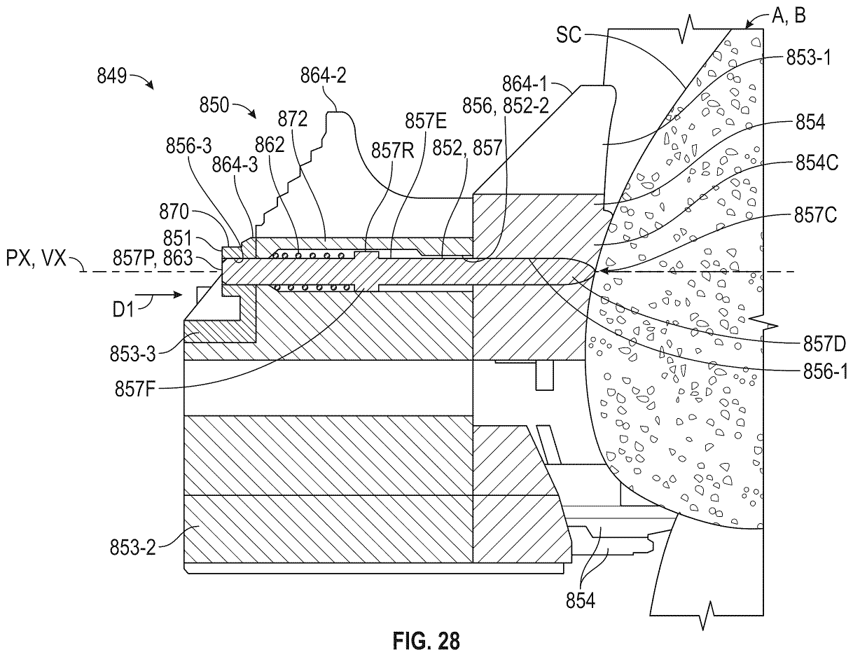

The guide body 453-1 and/or another portion of the transfer guide 450 may establish one or more passageways 456. The passageways 456 may include an opening in the patient-specific contact surface 450C. The passageway 456 may be adapted to receive an indicator body 457 of the verification member 452. In the implementation of FIG. 13, the receptacle 461 may be dimensioned such that a verification surface 457C of the indicator body 457 may be substantially flush with a rim of the opening when the locating component 464-1 is (e.g., correctly) seated on the bone B according to a respective surgical plan 133 (e.g., FIG. 2). In implementations, the contact surface 457C may be situated within the passageway 456 and/or may be substantially aligned with a distal opening of the passageway 456 when in a stopping position (see also FIG. 28). The indicator body 457 may be in an intermediate position relative to the carrier 458 when the locating component 464-1 is seated on the bone B according to the surgical plan 133.

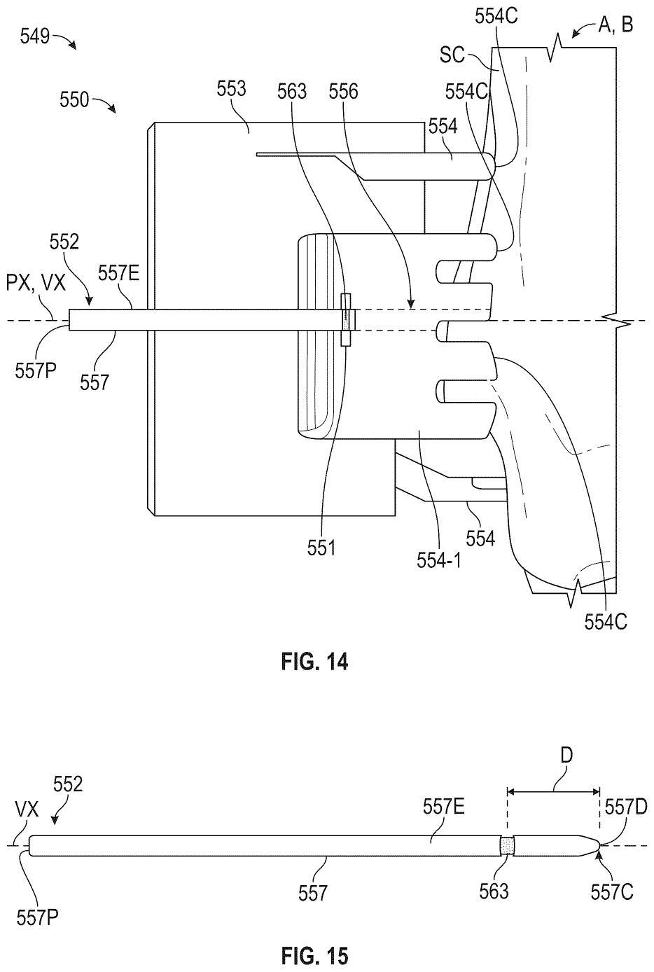

FIG. 14 discloses a surgical system 549 according to another implementation. The system 549 may include a transfer guide 550 and one or more position verification members 552. The guide 550 may include a guide body 553 and one or more locating members 554. Each locating member 554 may have one or more contact surfaces 554C. In implementations, a locating member 554-1 may have two or more (e.g., discontinuous) contact surfaces 554C, which may have respective patient-specific contours dimensioned to follow respective portions of the surface contour SC of the anatomy A. The guide body 553 may include one or more passageways 556 dimensioned to receive a respective verification member 552.

Referring to FIG. 15, with continuing reference to FIG. 14, the verification member 552 may be an elongated pin. The verification member 552 may be patient-generic. In other implementations, the verification member 552 may be patient-specific. The verification member 552 may be formed utilizing any of the techniques disclosed herein, such as machining to a predefined dimension (e.g., length). The verification member 552 may include a main (e.g., indicator) body 557 extending along a verification axis VX between a first (e.g., proximal) end 557P and a second (e.g., distal) end 557D. The verification member 552 may include one or more indicators 563. The indicator 563 may be established utilizing any of the techniques disclosed herein, such as a laser line. The indicator 563 may be established along a periphery 557E of the indicator body 557. The indicators 563 may be established at a distance D from the distal end 557D of the indicator body 557.

Referring back to FIG. 14, the guide body 557 may include one or more alignment features 551. The alignment feature 551 may be established based on a surgical plan 133 associated with the anatomy A of the patient (e.g., FIG. 2). The alignment feature 551 may be established based on the position of the indicator 563 along the verification member 552. The alignment feature 551 may have various geometries and may be established at various positions along the guide 550, such as a (e.g., proximal) face of the guide body 553. The alignment feature 551 may be an indicator (e.g., marking, hash mark or laser line) and/or an opening (e.g., cutout) in the guide 550. In the implementation of FIG. 14, the alignment feature 551 may include a slot. The alignment feature 551 may be established at a position along the passageway 556. The alignment feature 551 may have a dimension (e.g., width) that may be substantially equal to a dimension (e.g., width) of the indicator 563. The verification member 552 may be inserted through the passageway 556 and into contact with a surface contour SC of the respective bone B. The alignment feature 551 may be dimensioned such that the indicator 563 may be substantially aligned with the alignment feature 551 when the guide 550 may be (e.g., correctly) seated on the bone B according to the surgical plan 133.

The position verification members and/or associated component(s) may be formed according to a surgical plan 133 associated with a respective patient. The verification members may have a patient-specific geometry, including one or more patient-specific dimensions, which may be specified in the surgical plan 133. The verification members may be established based on a geometry of a respective surgical instrument (e.g., transfer guide) that may be adapted to receive or otherwise cooperate with the verification members. The patient-specific verification members may be formed utilizing any of the techniques disclosed herein.

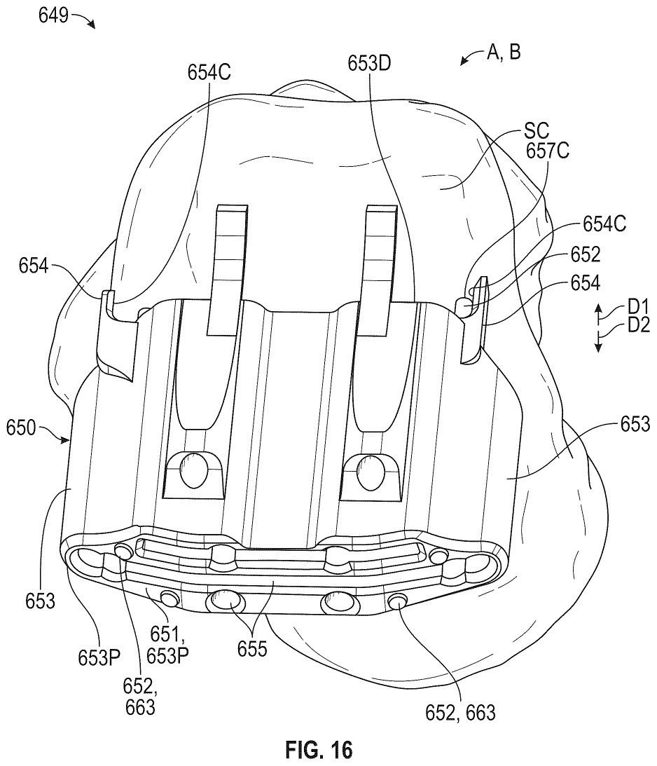

FIGS. 16-20 disclose a surgical system 649 according to an implementation. The system 649 may include a surgical instrument (e.g., transfer guide) 650 and one or more position verification members 652.

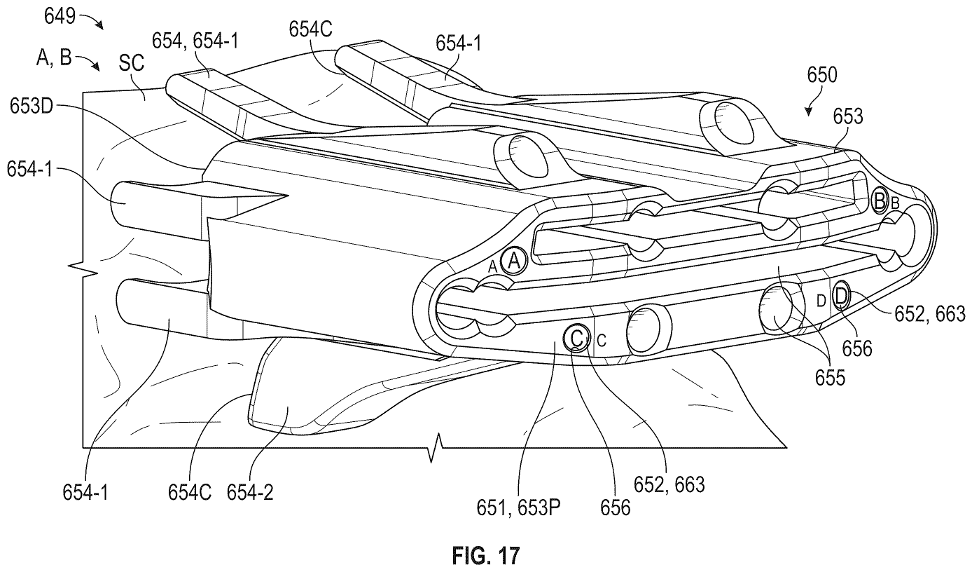

Referring to FIGS. 16-17, the guide 650 may include one or more patient-specific contact surfaces 654C dimensioned to seat the guide 650 in a patient-specific position and orientation on the anatomical surface of the anatomy A according to a surgical plan 133 (e.g., FIG. 2). The guide 650 may include one or more locating members 654 that may include respective patient-specific contact surfaces 654C. Each patient-specific contact surface 654C may be geometric or may include a patient-specific contour. The locating members 654 may include locating member(s) 654-1 and/or locating member(s) 654-2. The locating member(s) 654-1 may have one or more geometric contact surfaces 654C dimensioned to contact a surface contour SC of the anatomical surface according the surgical plan 133. The locating member(s) 654-2 may have one or more patient-specific contact surfaces 654C dimensioned to follow the surface contour SC according the surgical plan 133.

The verification members 652 may have one or more patient-specific dimensions, which may be the same or may differ from each other. The patient-specific dimension(s) may be established according to the surgical plan 133. The verification members 652 may be elongated pins. The verification member(s) 652 may be insertable through respective passageways 656 of the guide 650 to determine a placement (e.g., seating) of the guide 650 on an anatomical surface of the patient, such as a surface of the bone B.

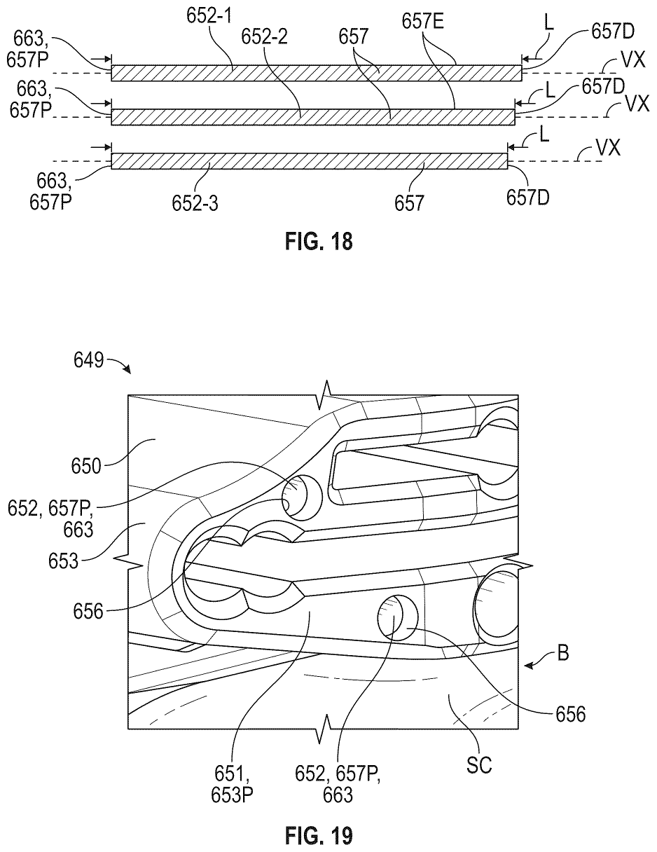

The dimensions and/or geometry of the verification members 652 may be the same or may differ. The verification members 652 may have different patient-specific dimensions (e.g., lengths) associated with a contour of the anatomical surface. In the implementation of FIG. 18, the system 649 may include a set of verification members 652 (e.g., 652-1, 652-2 and/or 652-3). Each verification member 652-1, 652-2, 652-3 may have a patient-specific length L. Each verification member 652 may include an indicator body 657 extending along a verification axis VX between a first (e.g., proximal) end 657P and a second (e.g., distal) end 657D, which may establish the respective length L. The verification members 652-1, 652-2, 652-3 may be insertable in respective passageway 656 in the guide body 653, which may be specified in the surgical plan 133. In other implementations, the verification members 652 may have the same dimension(s) (e.g., lengths) but may include respective indicators 663 established at different patient-specific positions along the respective verification members 652.

Each verification member 652 may be paired with a respective one of the passageways 656. The (e.g., unique) pairing(s) may be specified in the surgical plan 133. In the implementation of FIG. 17, the pairings of verification members 652 and passageways 656 may be identified by indicia (e.g., numbering or lettering).

The verification member 652 may be (e.g., independently) moveable in a plurality of positions relative to the passageway 656. The verification member 652 may be slidably received in the respective passageway 656 to establish a range of positions. The positions may be associated with different positions of the verification surface 657C relative to the distal end 653D of the guide body 653. In the implementation of FIG. 16, the verification surface 657C of the verification member 652 may be moveable in a first direction D1 away from the distal end 653D of the guide body 653. The verification surface 657C of the verification member 652 may be moveable in a second direction D2 toward the distal end 653D of the guide body 653. The surgeon may push or otherwise move the verification member 652 along the passageway 656 and into contact with the anatomy A.

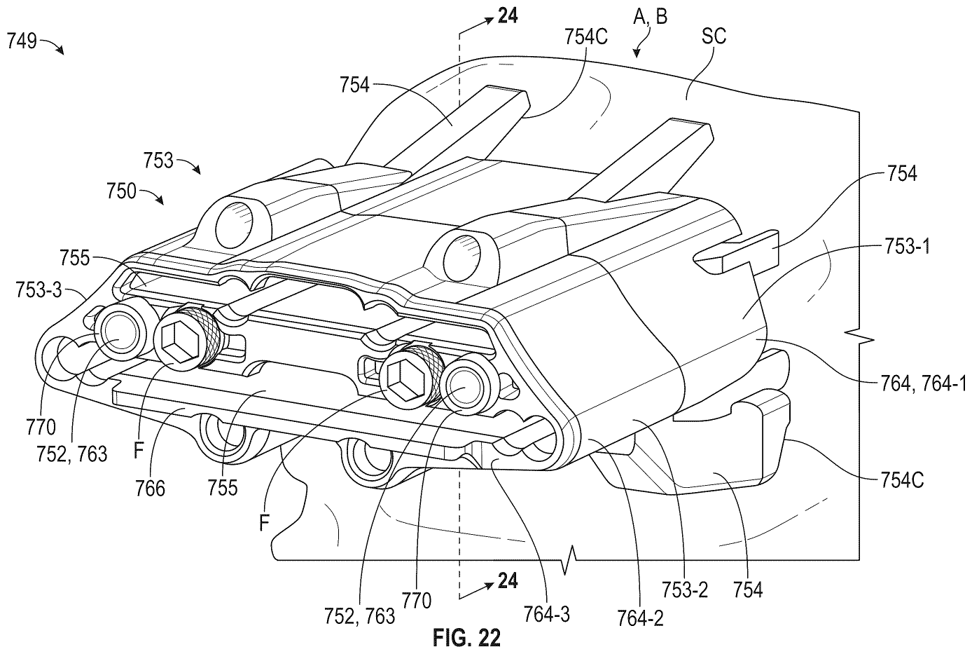

In the implementation of FIG. 21, two or more verification members 652′ may be fixedly attached or otherwise secured to each other to establish a verification device (e.g., assembly) 671′. In implementations, the verification members 652′ may be integrally formed to establish the verification device 671′. The verification members 652′ may be simultaneously positionable in respective passageways 656′ of a surgical instrument 650′. The surgical instrument 650′ may have one or more patient-specific contact surfaces 650C′, which may be dimensioned to substantially follow a surface contour SC of the anatomy A, such as a bone B. One or more of the passageways 656′ may have an opening along the respective contact surface 650C′. The surgeon may simultaneously position the verification members 652′ to check a placement (e.g., seating) of the instrument 650′, which may reduce interop time.

The verification member 652 may include one or more alignment features alignable with one or more alignment features of the guide 650 to indicate a placement (e.g., seating) of the guide 650 on the anatomical surface. The verification member 652 may include a first alignment feature that may be alignable with a second alignment feature of the guide 650 to indicate the placement. The alignment features may include any of the indicators and features disclosed herein.

The alignment feature(s) of the verification member 652 may include one or more indicators 663, including any of the indicators disclosed herein. The indicator (e.g., first alignment feature) 663 may be established along the proximal end 657P of the verification member 652.

The guide 650 may include one or more respective alignment features (e.g., indicators) 651. The alignment features 651 may be established utilizing any of the techniques disclosed herein. The (e.g., second) alignment feature 651 may be established along a face of the guide 650, such as an adjacent face along the proximal end 653P of the guide body 653.

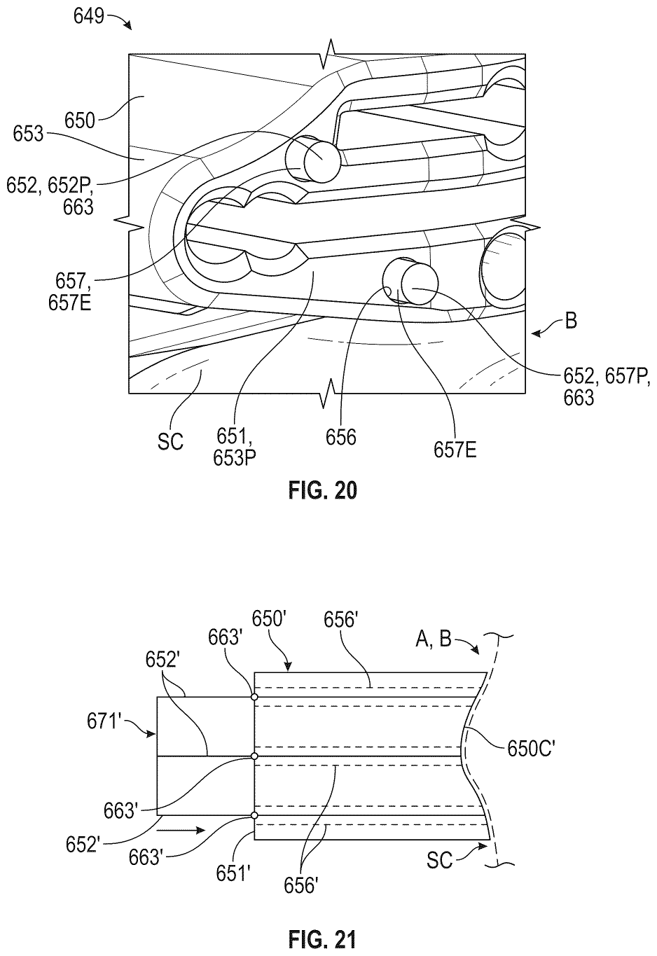

The indicator(s) 663 may be aligned with the respective alignment feature(s) 651 in response to the guide 650 and locating member(s) 654 being seated on the surface contour SC of the bone B in a single, patient-specific position and orientation, which may be specified in a surgical plan 133 (e.g., FIG. 2). In implementations, the verification member 652 may be dimensioned such that the indicator 663 and alignment feature 651 may be substantially aligned with each other in a single (e.g., intermediate) position (e.g., FIG. 17). Fewer than all indicator(s) 663 being aligned with the respective alignment feature(s) 651 may be associated with (e.g., indicate) an incorrect placement (e.g., seating) of the guide 650 and/or one or more locating member(s) 654. The verification member 652 may be dimensioned such that the proximal end 657P of the verification member 652 may be situated in the respective passageway 656 (e.g., FIG. 19) or may protrude from a (e.g., proximal) end of passageway 656 (e.g., FIGS. 16 and 20) in response to a placement (e.g., position and/or orientation) of the guide 650 and/or one or more of the locating member(s) 654 on the surface contour SC of the bone B differing from the surgical plan 133. The surgeon may reposition the guide 650 on the bone B and may check the alignment of the indicator(s) 663 to determine whether the guide 650 is correctly seated on the bone B according to the patient-specific position and orientation specified in the surgical plan 133. In the implementation of FIG. 21, indicators 263′ may be established along the respective verification members 652′. The indicators 263′ may be aligned with one or more respective alignment features 651′ of the instrument 650′ to indicate a placement (e.g., seating) of the instrument 650′ on the anatomy.

The planning environment 127 may be operable to establish a configuration 147 of the instrument (e.g., guide) 650/650′ and/or verification member(s) 652/652′ such that the indicator(s) 663/663′ may be substantially aligned with the respective alignment feature(s) 651/651′ when the guide 650/650′ may be positioned on the anatomy A according to the respective surgical plan 133. The configuration 147 may include dimension(s) and/or geometry of the guide 350/650 and/or verification member(s) 652/652′, a position and/or dimension(s) of the indicator(s) 663/663′ relative to the respective verification member(s) 652/652′, and/or a position and/or dimension(s) of the respective alignment feature(s).

FIGS. 22-24 disclose a surgical system (e.g., assembly) 749 according to another implementation. The system 749 may be utilized to perform various surgical procedures, including any of the procedures disclosed herein. The system 749 may include a surgical instrument (e.g., transfer guide or assembly) 750 and one or more position verification members 752. The guide 750 may be utilized with any of the position verification members disclosed herein. The guide 750 may include a guide body 753 adapted to position one or more surgical instruments.

The guide 750 may be modular and may include one or more patient-specific modules and/or one or more patient-generic (e.g., reusable) modules. The guide 750 may include one or more components (e.g., modules) 764. The guide 750 may include one or more patient-specific modules 764 and/or one or more patient-generic (e.g., reusable) modules 764. The modules 764 may be releasably secured or otherwise coupled to each other to establish a (e.g., modular) assembly. In implementations, the guide 750 may include a first (e.g., locating or positioning) module 764-1, a second (e.g., guide) module 764-2 and/or a third (e.g., depth stop) module 764-3. In other implementations, the first, second and/or third modules 764-1, 764-2, 764-3 may be combined with each other. In implementations, the first module 764-1 may be a patient-specific positioning (e.g., locating) module. The second module 764-2 may be a patient-generic (e.g., reusable) guide module. The third module 764-3 may be a patient-specific depth stop module. One or more of the modules 764 may be omitted, such as the depth stop module 764-3. The modules 764 may cooperate to establish respective portions 753-1, 753-2, 753-3 of the guide body 753.

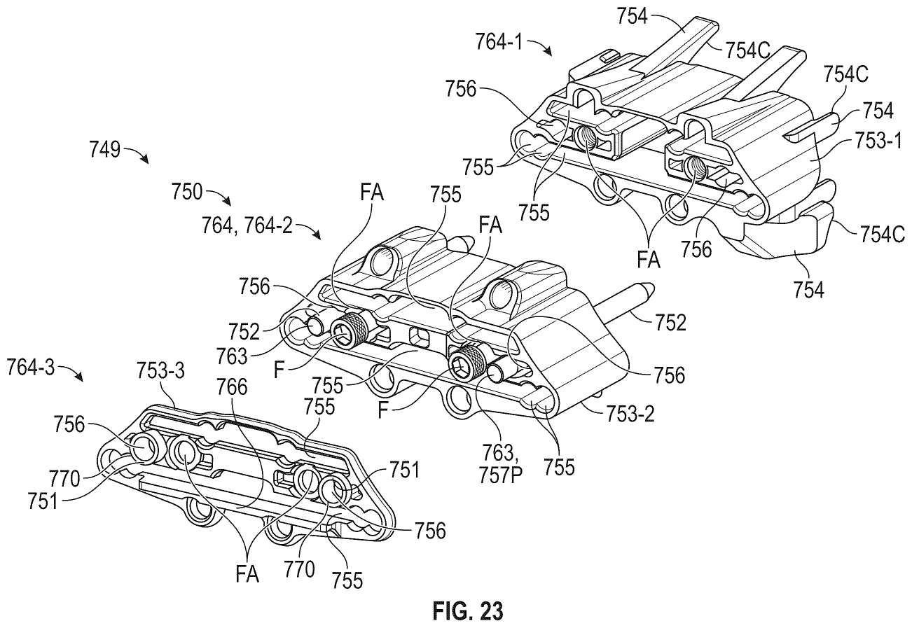

Various techniques may be utilized to secure the modules 764 to each other. The positioning module 764-1, the guide module 764-2 and/or depth stop module 764-3 may be releasably securable to each other. The modules 764 may be secured to together with one or more fasteners, clips, and/or interlocking connections. In implementations, the guide 750 may include one or more fasteners F. The fasteners F may be insertable in fixation apertures FA in the module(s) 764 (e.g., FIG. 23). The fastener F may have threading that may mate with threading along the aperture FA in at least the locating module 764-1 to secure the modules 764 to each other. The guide module 764-2 may be trapped or otherwise secured between the locating module 764-1 and depth stop 764-3 in an assembled configuration.

The locating module 764-1 and/or another portion of the guide 750 may include one or more patient-specific contact surfaces. The locating module 764-1 may include one or more locating members 754. The guide body 753-1 and locating members 754 may be established according to any of the techniques disclosed herein. The locating members 754 may include respective patient-specific contact surfaces 754C, which may be dimensioned to seat the guide 750 on an anatomical surface of an anatomy A of the patient in a patient-specific position and orientation. The patient-specific position and orientation may be established according to a surgical plan 133 for a respective patient (e.g., FIG. 2).

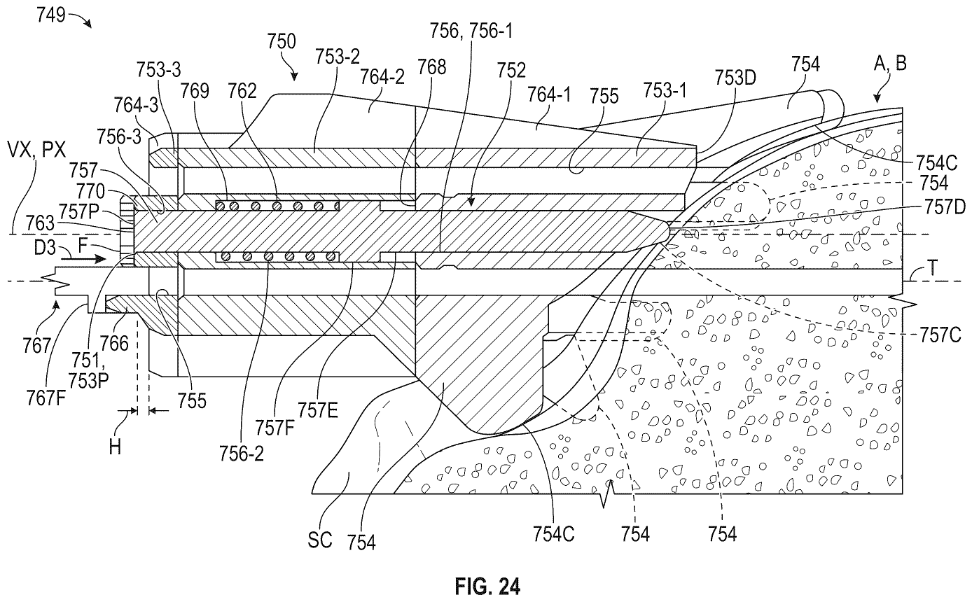

The guide module 764-2 may include one or more openings (e.g., guide apertures) 755. Each guide aperture 755 may be adapted to receive a surgical instrument, including any of the instruments disclosed herein such as a saw blade, drill or guide element (e.g., pin or wire). The guide apertures 755 may be established in the guide body 753-2 of the guide module 764-2. The guide apertures 755 may be aligned with respective guide apertures 755 of the locating module 764-1 and/or depth stop module 764-3 in an assembled configuration (e.g., FIGS. 22 and 24).

The depth stop module 764-3 may include one or more patient-specific depth stops 766 (e.g., standoffs). The depth stop 766 may extend from the guide body 753-3 of the depth stop module 764-3. The depth stop 766 may be dimensioned to limit insertion of surgical instrument(s) through the respective guide apertures 755 of the guide body 753. The depth stop 766 may be dimensioned according to the surgical plan 133.

Referring to FIG. 24, with continuing reference to FIGS. 22-23, the depth stop 766 may be dimensioned to limit insertion of a surgical instrument (e.g., saw blade or drill) 767 in a third (e.g., distal) direction D3 through the guide aperture (e.g., resection slot or drill passage) 755 in response to abutment between the depth stop 766 and an engagement feature (e.g., protrusion) 767F of the instrument 767. The depth stop 766 may be dimensioned based on a geometry of a bone B along a trajectory T of the respective guide aperture 755 when the guide 750 is seated on the bone B according to the surgical plan 133. The depth stop 766 may be useful in limiting trauma to the adjacent anatomy, including soft tissue.

The verification member(s) 752 may be positioned in one or more respective passageways 756. The passageway(s) may extend through the locating module 764-1, the guide module 764-2 and/or the depth stop module 764-3 in the assembled configuration. The modules 764-1, 764-2 and/or 764-3 may be dimensioned to establish respective (e.g., distal, intermediate and/or proximal) segments (e.g., sections) 756-1, 756-2 and/or 756-3 of the passageway(s) 756. The locating module 764-1 and guide module 764-2 may be (e.g., releasably) securable to each other to capture the verification member(s) 752 in the respective passageway(s) 756. In other implementations, at least the modules 764-1, 764-2 may form a monolithic structure. The verification member 752 and modules 764-1, 764-2 may be formed together by additive manufacturing (e.g., printing) such that the verification member 752 may be captured within the guide 750.

Referring to FIG. 24, with continuing reference to FIGS. 22-23, each verification member 752 may include a contact (e.g., verification) surface 757C adapted to contact tissue. The verification member 752 may be adapted to establish contact along a surface contour SC of the associated bone B.

The verification member(s) 752 may be moveable in a plurality of positions relative to the passageway(s) 756, including a starting position, a stopping position, and/or one or more intermediate positions. A location of the contact surface 757C of the verification member 752 in the starting position may be distal of a location of the contact surface 757C in the stopping position relative to a passage axis PX of the passageway 756. The starting position may be associated with a distal-most position of the contact surface 757C of the verification member 752 relative to the guide 750 and/or passage axis PX when the verification member 752 may be situated in the passageway 756. The stopping position may be associated with a proximal-most position of the contact surface 757C relative to the guide 750 and/or passage axis PX when the verification member 752 may be situated in the passageway 756.

The verification member(s) 752 may be (e.g., releasably) securable to the guide 750. Various techniques may be utilized to secure (e.g., capture) the verification member(s) 752. In the implementation of FIG. 24, the verification member 752 may include an indicator body 757. The verification member 752 may include a protrusion (e.g., flange) 757F, which may extend from a periphery 757E of the indicator body 757. The intermediate segment 756-2 of the passageway 756 may be established between opposed first and second (e.g., distal and proximal) walls 768, 769. The locating module 764-1 may include the distal wall 768. The distal wall 768 may be established by a (e.g., proximal) face of the locating module 764-1. The proximal wall 769 may be established by the guide module 764-2. The flange 757F may be moveable along a length of the intermediate segment 756-2 of the passageway 756. The distal wall 768 and/or proximal wall 769 may be adapted to abut the flange 757F to limit the starting position and/or stopping position of the verification member 752 and/or associated contact surface 757C.

A spring member 762 may be arranged intermediate segment 756-2 of the passageway 756. The spring member 762 may be captured between the flange 757F and the proximal wall 769. The spring member 762 may be adapted to bias the verification member 752 distally from the passageway 756. The spring member 762 may bias the verification member 752 toward the bone B when the guide 750 is seated on the bone B. The spring member 762 may cause the contact surface 757C of the verification member 752 to establish contact with the surface contour SC of the bone B.

Each verification member 752 may include one or more indicators 763. The indicator(s) 763 may be established utilizing any of the techniques disclosed herein. The guide 750 may include one or more alignment features 751, which may be associated with the respective passageway(s) 756. In implementations, the alignment features 751 may be a face of the guide 750, which may be established along or otherwise adjacent to the proximal end 753P of the guide body 753.

The guide 750 may include one or more (e.g., patient-specific) standoffs 770. In implementations, the depth stop module 764-3 may include the standoff(s) 770. The standoffs 770 may establish respective alignment features 751. The geometry of the standoffs 770 may be the same or may differ from each other. The standoffs 770 may be aligned with, or may otherwise be adjacent to, one or more of the passageways 756 through the guide body 753. The standoffs 770 may include respective patient-specific heights H. The heights H of the standoffs 770 may be the same or may differ from each other. The heights H may be established based on the surface contour SC of the bone B when the guide 750 is seated on the bone B according to the surgical plan 133.

The verification member 752 may be insertable through the respective passageway 756 to indicate whether the locating module 764-1 may be seated on the anatomical surface of the anatomy A in a patient-specific position and orientation according to the surgical plan 133 based on a position of the indicator 763 relative to the alignment feature 751 of the respective standoff 770 when the verification member 752 is in contact the anatomical surface.

The indicator 763 may be established along a first (e.g., proximal) end 757P of the verification member 752. A (e.g., proximal) surface of the standoff 770 may establish the alignment feature 751. The standoff 770 may be dimensioned such that the proximal end 757P of the verification member 752 may be substantially aligned with the respective alignment feature 751 when the guide 750 and/or associated locating member(s) 754 are positioned and oriented on the surface contour SC of the bone B according to the surgical plan 133. The proximal end 757P of the verification member 752 may be substantially flush with the proximal end of the respective standoff 770 and/or a (e.g., proximal) rim of the respective passageway 756 in response to positioning at least an adjacent portion of the guide 750 on the anatomical surface in the patient-specific position and orientation. The standoff 770 may be dimensioned such that the proximal end 757P of the verification member 752 may be substantially flush with the proximal end of the standoff 770 in the intermediate position of the verification member 752.

The verification member 752 may be dimensioned such that the proximal end 757P and/or indicator 763 may be situated in the respective passageway 756 in the starting position. The verification member 752 may be dimensioned such that the proximal end 757P and/or indicator 763 may protrude from the proximal end of passageway 756 in the stopping position. In other implementations, the verification member 752 may be dimensioned such that the proximal end 757P and/or indicator 763 may be substantially aligned with the proximal end of the standoff 770 and/or alignment feature 751 in the stopping position. The verification member 752 may be dimensioned such that the proximal end 757P and/or indicator 763 may be situated in the respective passageway 756 (see, e.g., FIG. 19) or may protrude from a (e.g., proximal) end of passageway 756 (see, e.g., FIG. 20) in response to a placement (e.g., position and/or orientation) of the guide 750 and/or one or more of the locating member(s) 754 on the anatomical surface of the anatomy A differing from the surgical plan 133. Permitting the verification member 752 to move proximally past the patient-specific position relative to the passage axis PX may reduce a likelihood that the verification member 752 may inhibit the guide 750 from being correctly positioned on the anatomy A, which may otherwise be caused by a limiting a travel of the verification member 752 to the patient-specific position associated with the indicator 763.

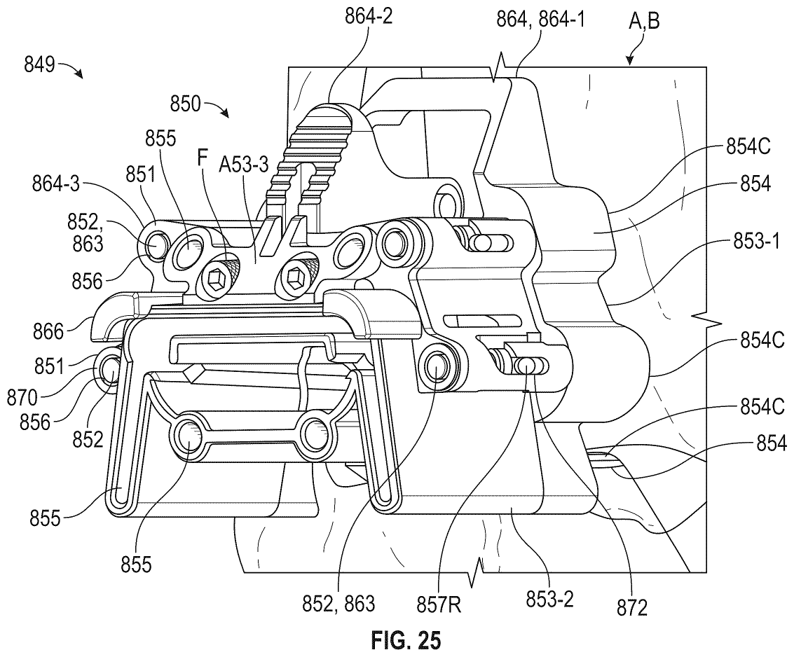

FIGS. 25-29 disclose a surgical system (e.g., assembly) 849 according to another implementation. The system 849 includes a surgical instrument (e.g., transfer guide) 850 and one or more position verification members 852. The guide 850 may include one or more components (e.g., modules) 864, such as a first (e.g., positioning or locating) module 864-1, a second (e.g., guide) module 864-2 and/or a third (e.g., depth stop) module 864-3. The modules 864 may be secured to each other utilizing any of the techniques disclosed herein. The depth stop module 864-3 may include one or more depth stops 866.

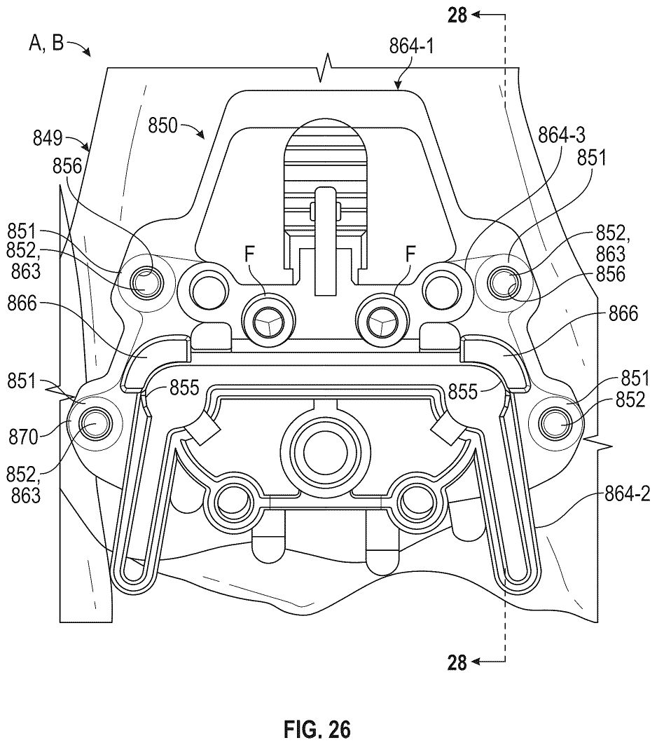

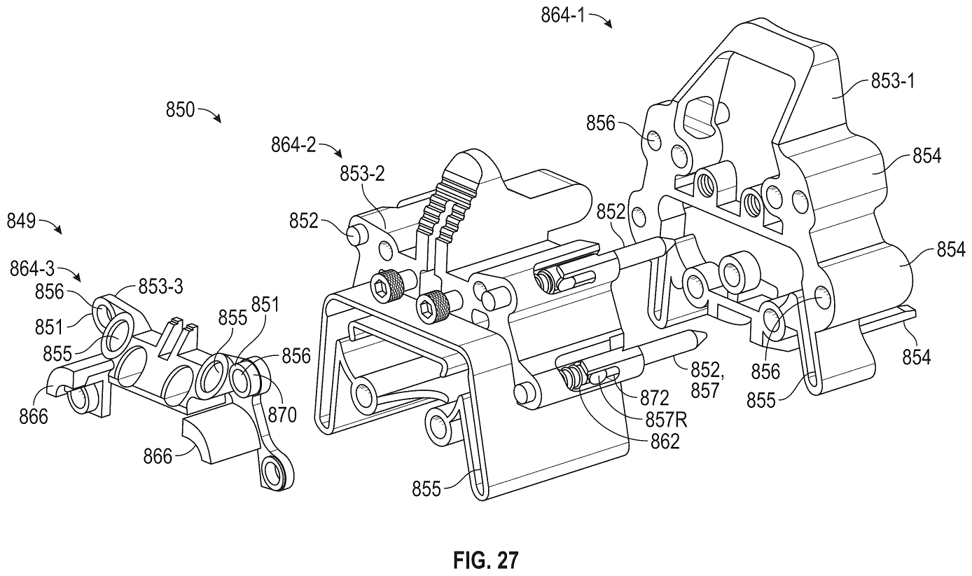

Referring to FIGS. 27-29, with continuing reference to FIGS. 25-26, the verification members 852 may include one or more elongated pins. The verification members 852 may include a main (e.g., indicator) body 857. The verification member 852 may include a flange 857F, which may extend from the indicator body 857. A spring member 862 may be dimensioned to engage the flange 857F to bias the verification member 852 in a first (e.g., distal) direction D1 (FIG. 28).

The verification member 852 may be releasably secured to the guide 850. In implementations, the verification member 852 may include a protrusion (e.g., key) 857R. The protrusion 857R may extend from a periphery 857E of the indicator body 857. In implementations, the protrusion 857R may extend outwardly from the flange 857F.

A guide body 853-2 of the guide module 864-2 may include a channel (e.g., keyway) 872. The channel 872 may extend from a respective passageway 856. In the implementation of FIG. 28, the channel 872 may be established along an intermediate segment 856-2 of the passageway 856. The channel 872 may be dimensioned to mate with the protrusion 857R of the respective verification member 852 situated in the passageway 856 to limit axial and/or rotational movement of the verification member 852 relative to a passage axis PX of the passageway 856.

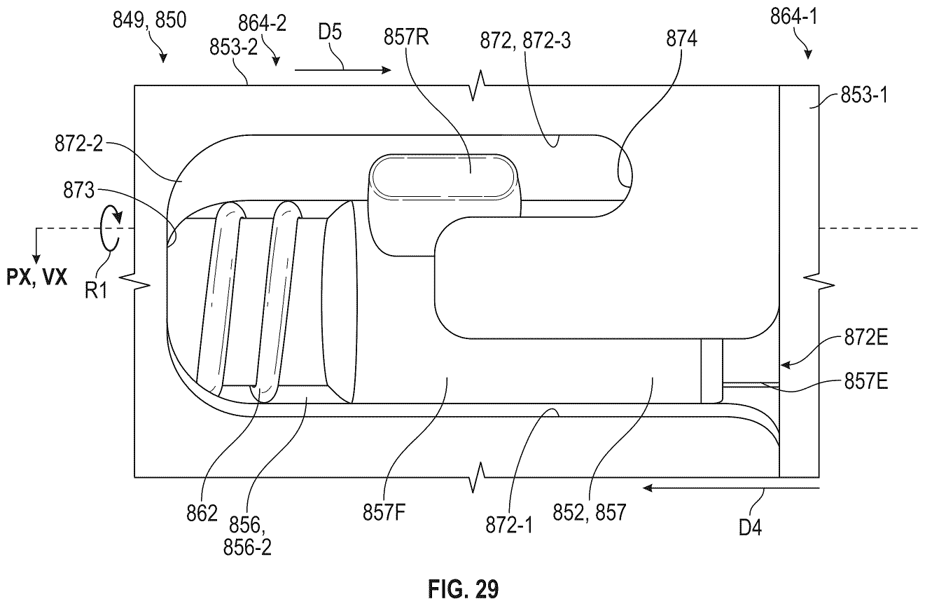

The channel 872 may have various geometries. Referring to FIG. 29, with continuing reference to FIGS. 25-28, the channel 872 may have an L-shaped or J-shaped geometry. The channel 872 may include first, second and/or third segments 872-1, 872-2 and/or 872-3. The first and/or third segments 872-1, 872-3 may extend lengthwise along the respective passageway 856 relative to the passage axis PX. The second segment 872-2 may extend between the first and third segments 872-1, 872-3. The second segment 872-2 may extend circumferentially about the passage axis PX to interconnect the first and third segments 872-1, 872-3. The protrusion 857R may be translatable along the segments 872-1, 872-2 and/or 872-3. In other implementations, the third segment 872-3 may be omitted.

The channel 872 may be dimensioned such that the verification member 852 may be releasably securable in the passageway 856 in response to moving the protrusion 857R in a fourth (e.g., proximal) direction D4 along the first segment 872-1, and then rotating the verification member 852 in a first rotational direction R1 to cause the protrusion 857R to move along the second segment 872-2. In implementations, the channel 872 may be dimensioned such that the verification member 852 may be releasably securable in the passageway 856 in response to moving the protrusion 857R in a fifth (e.g., distal) direction D5 along the third segment 872-3. The fifth direction D5 may be opposed to the fourth direction D4. The spring member 862 may be dimensioned to bias the verification member 852 in the fifth direction D5. The verification member 852 may be releasable from the passageway 856 in response to moving the verification member 852 and protrusion 857R in an opposite order. A first (e.g., proximal) wall 873 along the second segment 872-2 may be dimensioned to bound the stopping position of the verification member 852 in response to abutment between the wall 873 and the protrusion 857R. A second (e.g., distal) wall 874 along a terminal end of the third segment 872-3 may be dimensioned to bound the starting position of the verification member 852 in response to abutment between the wall 874 and the protrusion 857R. A main body 853-1 of the positioning module 864-1 may be dimensioned to abut a main body 853-2 of the guide module 864-2 to block an entrance 872E to the channel 872. The entrance 872E may be established along the first segment 872-1.

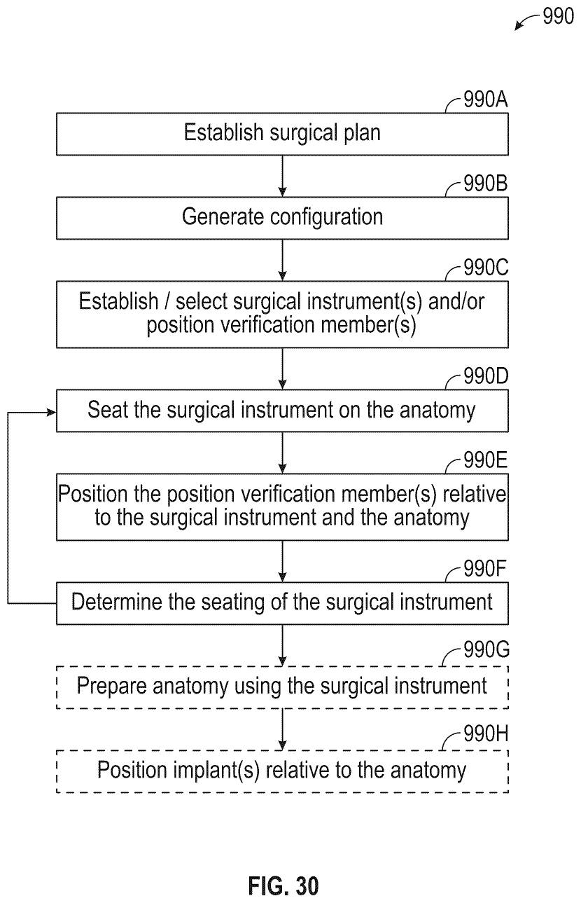

FIG. 30 discloses a method of performing a surgical procedure in a flowchart 990. The method may be utilized to perform various orthopaedic procedures such as an arthroplasty for restoring functionality to various bones and joints, including any of the bones and associated joints disclosed herein such as shoulder, ankle, hip, knee and elbow joints. The method may be utilized with any of the surgical instruments (e.g., transfer guides) and/or position verification members disclosed herein. Fewer or additional steps than are recited below could be performed within the scope of this disclosure, and the recited order of steps is not intended to limit this disclosure. The planning environment 127 and any of the associated modules may be configured to perform any of the functionality of the method 990. Reference is made to the system 120, surgical instrument (e.g., transfer guide) 250 and position verification members 252.

Referring to FIG. 2, with continuing reference to FIG. 30, at block 990A a surgical plan 133 for a patient may be established. Block 990 may include selecting one or more implants for positioning on an anatomy of the patient. The surgeon or clinical user may select the implant and/or an associated implant type, size and/or configuration. The implant(s) may be specified in the surgical plan 133.

At block 990B, one or more configurations 147 may be generated based on the surgical plan 133. The configuration(s) 147 may be associated with respective transfer model(s) 148.

Referring to FIGS. 4-5, with continuing reference to FIGS. 2 and 30, at block 990C one or more surgical instruments (e.g., transfer guides) 250 and/or position verification member(s) 252 may be established and/or selected. The transfer guide 250 and verification member(s) 252 may be established utilizing any of the techniques disclosed herein. The transfer guide and the verification member(s) may be patient-specific and/or patient-generic. The transfer guide may be patient-specific and the verification members may be patient-generic, or vice versa. A geometry of the transfer guide 250 may be established based on the configuration 147. In implementations, a geometry of the position verification member(s) may be established based on the configuration 147 (e.g., verification members 652).

At block 990D, the transfer guide 250 may be seated on an anatomical surface of a patient. The anatomical surface may include a surface contour SC of a respective bone B. The guide 250 may be seated on the anatomical surface utilizing any of the techniques disclosed herein. The guide 250 may be seated on the anatomical surface based on the surgical plan 133. Block 990D may include positioning the contact surfaces 254C of the locating member(s) 254 of the guide 250 in contact with the surface contour SC of the bone B at respective contact points CP, which may be specified by the surgical plan 133.

The guide 250 may include one or more patient-specific locating members 254 having respective contact surfaces 254C. The contact surfaces 254C may be dimensioned to seat the guide 250 on the anatomical surface of the anatomy A in the patient-specific placement (e.g., position and/or orientation). Placing (e.g., seating) the guide 250 may include establishing contact between the patient-specific locating member(s) 254 and the anatomical surface at the respective contact points CP, which may correspond to contact points specified in the surgical plan 133 (e.g., contact points CP of FIG. 3). In implementations, the contact surface 254C of at least one or more of the patient-specific locating members 254 may be dimensioned to follow a surface contour SC of the anatomical surface of the anatomy A, such as a respective bone B. In implementations, the contact surface 254C may include an opening to the respective passageway 256 (e.g., passageways 456 and 856 of FIGS. 13 and 28).

At block 990E, the verification member(s) 252 may be positioned relative to the guide 250 and/or the anatomy A. The verification member(s) 252 may be positioned relative to the guide 250 prior to, during, and/or subsequent to placing the guide 250 on the anatomy A. In the implementation of FIGS. 16-20, the verification member(s) 652 may be patient-specific. The verification member(s) 652 may have respective patient-specific length(s) L based on the surgical plan 133 (e.g., FIG. 18). In implementations, block 990E may include inserting a set of the position verification members 652 through respective passageways 656 of the guide 650. The verification members 652 may have different patient-specific lengths L based on the surface contour SC of the anatomical surface of the anatomy A.

Block 990E may include positioning the verification (e.g., contact) surface(s) 257C of the verification member(s) 252 in contact with the surface contour SC of the bone B at respective contact points CP (e.g., FIGS. 8-9). Block 990E may include positioning the verification member 252 through respective passageway 256 of the guide 250 such that the verification member 252 may extend distally from the passageway 256 to contact the anatomical surface at the respective contact point CP (e.g., verification member 652 of FIG. 17). The verification member 252 may be biased distally towards the anatomical surface of the anatomy A. The verification member 252 may include an indicator 263 moveable in a plurality of positions, which may be relative to a passage axis PX of the respective passage 256. The positions may include a starting position, a stopping position and an intermediate position between the starting and stopping positions. In the implementation of FIGS. 16-17 and 19-20, block 990E may include positioning the patient-specific verification members 652 through the respective passageway(s) 656 of the guide 650 to contact the anatomical surface of the anatomy A.

At block 990F, the method may include determining a placement (e.g., seating) of the guide 250 relative to the anatomy A, which may be based on the surgical plan 133. Block 990F may include determining that the guide 250 may be seated in a patient-specific placement (e.g., position and/or orientation) specified by the surgical plan 133 based on the position(s) of the indicator(s) 263. Block 990F may include determining that the guide 250 may be seated in the patient-specific placement specified by the surgical plan 133 in response to the indicator(s) 263 being aligned with respective alignment feature(s) in a preselected (e.g., intermediate) position, but determining that the placement of the guide 250 may deviate from the patient-specific placement (e.g., position and/or orientation) in response to the indicator(s) 263 being misaligned with the alignment feature(s). In implementations, block 990F may include determining that the guide 250 may be (e.g., correctly) seated in the patient-specific placement according to the surgical plan 133 in response to all, or at least a majority, of the indicator(s) 263 being aligned with the respective alignment feature(s).

The alignment feature(s) may be established utilizing any of the techniques disclosed herein. The alignment feature(s) may be established by another portion of the verification member 252, such as the carrier 258. In implementations, the alignment feature(s) may be established by the guide 250 (e.g., alignment features 551, 651, 751, 851 of FIGS. 14, 17, 23 and 26). In the implementation of FIGS. 16-17 and 19-20, block 990F may include determining whether the guide 650 may be seated in a patient-specific placement (e.g., position and/or orientation) specified by the surgical plan 133 based on a position of the verification member(s) 652 relative to the guide 650. In the implementation of FIG. 20, a proximal end 656P of the verification member 652 may be within the passageway 656 and/or may protrude from a proximal end of the passageway 656 in response to the placement of the guide 650 deviating from the surgical plan 133.

One or more iterations of blocks 990D, 990E and/or 990F may be performed in response to determining that the placement of the guide 250 may deviate from the surgical plan 133. The surgeon may move the guide 250 from a first position to a second, different position on the anatomy A. The surgeon may recheck the verification member(s) 652 to determine whether the guide 250 may be correctly seated on the anatomy A.

At block 990G, the anatomy A may be prepared using the guide 250. Block 990G may include fixedly attaching or otherwise securing the guide 250 to the bone B, which may occur subsequent to determining that the guide 250 is (e.g., correctly) seated on the anatomy A according to the surgical plan 133 at block 990F. The guide 250 may be secured to the bone B utilizing one or more fixation elements (e.g., pins or K-wires), which may be driven through respective guide apertures 255 and into bone.

Block 990G may include guiding one or more surgical tools through the guide aperture(s) 255 to engage the anatomical surface of the anatomy A. The surgical tools may include any of the surgical instruments and devices disclosed herein, such as a saw blade, drill and/or guide element. The surgical tool may be adapted to remove tissue from the anatomy A. Block 990G may include removing the guide 250 and/or verification members 252 from the surgical site.

At block 990H, one or more implants may be positioned relative to the anatomy A. The implants may include any of the implants disclosed herein and may be associated with a prosthesis. The implant(s) may be specified in the surgical plan 133. The implant(s) may be secured to the bone B and may restore functionality to the bone B and/or an associated joint.

The novel devices and methods of this disclosure provide improved accuracy in positioning surgical instruments (e.g., transfer guides) relative to an anatomy of the patient. The placement of the instrument may be specified by a surgical plan for the patient. One or more position verification members may be adapted to determine whether the instrument is (e.g., correctly) seated on the anatomy based on the surgical plan. The verification member may be adapted to indicate a degree of fit between the instrument and the anatomical surface. The surgical instruments and/or position verification members may be patient-generic and/or patient-specific. Increased accuracy in the placing (e.g., seating) the instrument may improve the surgical outcome, including improved preparation of the anatomy. In implementations, the verification member may be secured to the surgical instrument, which may decrease interoperative time that may otherwise be incurred due to assembly.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. An assembly for a surgical procedure comprising:
a guide including a guide body dimensioned to guide a surgical instrument, wherein the guide body includes one or more patient-specific contact surfaces and one or more passageways; and
one or more position verification members receivable in a respective one of the passageways, the position verification member including at least one indicator and a verification surface adapted to contact tissue;
wherein the at least one indicator is moveable in a plurality of positions relative to the guide body in response to translation of the respective position verification member along the respective passageway, the positions include a starting position, a stopping position and an intermediate position between the starting and stopping positions, and the intermediate position is associated with a predetermined placement of the one or more patient-specific contact surfaces relative to an anatomical surface of a patient.

2. The assembly as recited in claim 1, wherein:
the one or more passageways include a first passageway having an opening along the respective patient-specific contact surface.

3. The assembly as recited in claim 1, wherein a spring member is dimensioned to bias the respective position verification member toward the starting position.

4. (canceled)

5. The assembly as recited in claim 3, wherein the spring member is captured between the guide body and a flange of the respective position verification member.

6. The assembly as recited in claim 1, wherein the position verification member includes an elongated pin extending between a proximal end and a distal end.

7. (canceled)

8. The assembly as recited in claim 6, wherein:
the elongated pin includes a patient-specific length.

9-11. (canceled)

12. The assembly as recited in claim 6, wherein:
the position verification member includes a carrier having a carrier passage dimensioned to receive the elongated pin, and the elongated pin is moveable relative to the carrier; and
the carrier is adapted to engage the guide body when the elongated pin is inserted through the respective passageway.

13. The assembly as recited in claim 12, wherein:
the carrier includes at least one viewing aperture along a periphery of the carrier passage; and
the at least one indicator includes a first indicator at least partially alignable with the at least one viewing aperture to indicate the position of the verification surface.

14-15. (canceled)

16. The assembly as recited in claim 12, wherein:
the guide body includes a receptacle extending from a proximal end of the passageway, and the receptacle includes a patient-specific depth; and
the carrier is at least partially receivable in the receptacle to set the position of the elongated pin relative to the guide body.

17. The assembly as recited in claim 6, wherein:
the position verification member includes a protrusion extending from a periphery of the elongated pin;
the guide body includes a J-shaped channel including first, second and third segments, the first and third segments extend lengthwise along the respective passageway, and the second segment extends circumferentially about the respective passageway to interconnect the first and third segments; and
the elongated pin is releasably securable in the respective passageway in response to moving the protrusion in a first direction along the first segment, then rotating the elongated pin in a second direction to cause the protrusion to move along the second segment, and then moving the protrusion in a third direction along the third segment.

18. (canceled)

19. The assembly as recited in claim 17, further comprising:
one or more patient-specific standoffs adjacent the respective one or more passageways, wherein the standoff is dimensioned such that the proximal end of the respective elongated pin is substantially flush with a proximal end of the standoff in the intermediate position.

20. (canceled)

21. The assembly as recited in claim 1, wherein:
the guide body includes a first module and a second module releasably securable to each other to capture the position verification member in the respective passageway.

22. The assembly as recited in claim 21, wherein:
the first module comprises the one or more patient-specific contact surfaces.

23. The assembly as recited in claim 21, wherein:
the at least one position verification member includes a flange;
the passageway includes an intermediate section between opposed first and second walls, the flange is moveable along a length of the intermediate section, and the first and second walls adapted to abut the flange to limit the starting position and the stopping position; and
the first module comprises the first wall, and the second module comprises the second wall.

24. An assembly for a surgical procedure comprising:
a guide including a guide body dimensioned to position a surgical instrument, the guide body established by a locating module and a depth stop module, the depth stop module including one or more standoffs aligned with one or more passageways through the guide body, each standoff including a respective patient-specific height; and
one or more position verification members including a respective indicator and a verification surface adapted to contact tissue, the position verification member insertable through the respective passageway to indicate whether the locating module is positioned on an anatomical surface of a patient in a patient-specific position according to a surgical plan based on a position of the indicator relative to the respective standoff.

25. The assembly as recited in claim 24, wherein:
the guide includes at least one locating member having a patient-specific surface dimensioned to seat the guide on the anatomical surface in the patient-specific position.

26-34. (canceled)

35. A surgical kit comprising:
a surgical instrument; and
one or more position verification members having a respective patient-specific dimension according to a surgical plan, wherein the one or more position verification members are insertable through respective passageways of the surgical instrument to determine a placement of the surgical instrument on an anatomical surface of a patient.

36. The kit as recited in claim 35, wherein:
the position verification members have different patient-specific lengths associated with a contour of the anatomical surface.

37. (canceled)

38. The kit as recited in claim 35, wherein:
the one or more position verification members includes a first alignment feature that is alignable with a second alignment feature of the surgical instrument to indicate the placement of the surgical instrument on the anatomical surface.

39. (canceled)

40. The kit as recited in claim 35, wherein:
the surgical instrument includes one or more patient-specific contact surfaces dimensioned to seat the surgical instrument in a patient-specific position on the anatomical surface according to the surgical plan.

41-51. (canceled)
